SEQUENCE LISTING (10 sequences)

SEQ ID NO: 1 - protein, 546 aa, Artificial Sequence
  1-Isoform Alpha-1
MTPLVSRLSRLWAIMRKPRAAVGSGHRKQAASQEGRQKHAKNNSQAKPSACDGMIAECPGAPAGLARQPEEVVLQASVSSYHLFRDVAEVTAFRGSLLSWYDQEKRDLPWRRRAEDEMDLDRRAYAVWVSEVMLQQTQVATVINYYTGWMQKWPTLQDLASASLEEVNQLWAGLGYYSRGRRLQEGARKVVEELGGHMPRTAETLQQLLPGVGRYTAGAIASIAFGQATGVVDGNVARVLCRVRAIGADPSSTLVSQQLWGLAQQLVDPARPGDFNQAAMELGATVCTPQRPLCSQCPVESLCRARQRVEQEQLLASGSLSGSPDVEECAPNTGQCHLCLPPSEPWDQTLGVVNFPRKASRKPPREESSATCVLEQPGALGAQILLVQRPNSGLLAGLWEFPSVTWEPSEQLQRKALLQELQRWAGPLPATHLRHLGEVVHTFSHIKLTYQVYGLALEGQTPVTTVPPGARWLTQEEFHTAAVSTAMKKVFRVYQGQQPGTCMGSKRSQVSSPCSRKKPRMGQQVLDNFFRSHISTDAHSLNSAAQ

SEQ ID NO: 2 - protein, 536 aa, Artificial Sequence
  1-Isoform Alpha-2
MTPLVSRLSRLWAIMRKPRAAVGSGHRKQAASQEGRQKHAKNNSQAKPSACDAGLARQPEEVVLQASVSSYHLFRDVAEVTAFRGSLLSWYDQEKRDLPWRRRAEDEMDLDRRAYAVWVSEVMLQQTQVATVINYYTGWMQKWPTLQDLASASLEEVNQLWAGLGYYSRGRRLQEGARKVVEELGGHMPRTAETLQQLLPGVGRYTAGAIASIAFGQATGVVDGNVARVLCRVRAIGADPSSTLVSQQLWGLAQQLVDPARPGDFNQAAMELGATVCTPQRPLCSQCPVESLCRARQRVEQEQLLASGSLSGSPDVEECAPNTGQCHLCLPPSEPWDQTLGVVNFPRKASRKPPREESSATCVLEQPGALGAQILLVQRPNSGLLAGLWEFPSVTWEPSEQLQRKALLQELQRWAGPLPATHLRHLGEVVHTFSHIKLTYQVYGLALEGQTPVTTVPPGARWLTQEEFHTAAVSTAMKKVFRVYQGQQPGTCMGSKRSQVSSPCSRKKPRMGQQVLDNFFRSHISTDAHSLNSAAQ

SEQ ID NO: 3 - protein, 535 aa, Artificial Sequence
  1-Isoform Alpha-3
MTPLVSRLSRLWAIMRKPRAAVGSGHRKQAASQEGRQKHAKNNSQAKPSACDGLARQPEEVVLQASVSSYHLFRDVAEVTAFRGSLLSWYDQEKRDLPWRRRAEDEMDLDRRAYAVWVSEVMLQQTQVATVINYYTGWMQKWPTLQDLASASLEEVNQLWAGLGYYSRGRRLQEGARKVVEELGGHMPRTAETLQQLLPGVGRYTAGAIASIAFGQATGVVDGNVARVLCRVRAIGADPSSTLVSQQLWGLAQQLVDPARPGDFNQAAMELGATVCTPQRPLCSQCPVESLCRARQRVEQEQLLASGSLSGSPDVEECAPNTGQCHLCLPPSEPWDQTLGVVNFPRKASRKPPREESSATCVLEQPGALGAQILLVQRPNSGLLAGLWEFPSVTWEPSEQLQRKALLQELQRWAGPLPATHLRHLGEVVHTFSHIKLTYQVYGLALEGQTPVTTVPPGARWLTQEEFHTAAVSTAMKKVFRVYQGQQPGTCMGSKRSQVSSPCSRKKPRMGQQVLDNFFRSHISTDAHSLNSAAQ

SEQ ID NO: 4 - protein, 532 aa, Artificial Sequence
  1-Isoform Beta-1
MRKPRAAVGSGHRKQAASQEGRQKHAKNNSQAKPSACDGMIAECPGAPAGLARQPEEVVLQASVSSYHLFRDVAEVTAFRGSLLSWYDQEKRDLPWRRRAEDEMDLDRRAYAVWVSEVMLQQTQVATVINYYTGWMQKWPTLQDLASASLEEVNQLWAGLGYYSRGRRLQEGARKVVEELGGHMPRTAETLQQLLPGVGRYTAGAIASIAFGQATGVVDGNVARVLCRVRAIGADPSSTLVSQQLWGLAQQLVDPARPGDFNQAAMELGATVCTPQRPLCSQCPVESLCRARQRVEQEQLLASGSLSGSPDVEECAPNTGQCHLCLPPSEPWDQTLGVVNFPRKASRKPPREESSATCVLEQPGALGAQILLVQRPNSGLLAGLWEFPSVTWEPSEQLQRKALLQELQRWAGPLPATHLRHLGEVVHTFSHIKLTYQVYGLALEGQTPVTTVPPGARWLTQEEFHTAAVSTAMKKVFRVYQGQQPGTCMGSKRSQVSSPCSRKKPRMGQQVLDNFFRSHISTDAHSLNSAAQ

SEQ ID NO: 5 - protein, 522 aa, Artificial Sequence
  1-Isoform Gamma-2
MRKPRAAVGSGHRKQAASQEGRQKHAKNNSQAKPSACDAGLARQPEEVVLQASVSSYHLFRDVAEVTAFRGSLLSWYDQEKRDLPWRRRAEDEMDLDRRAYAVWVSEVMLQQTQVATVINYYTGWMQKWPTLQDLASASLEEVNQLWAGLGYYSRGRRLQEGARKVVEELGGHMPRTAETLQQLLPGVGRYTAGAIASIAFGQATGVVDGNVARVLCRVRAIGADPSSTLVSQQLWGLAQQLVDPARPGDFNQAAMELGATVCTPQRPLCSQCPVESLCRARQRVEQEQLLASGSLSGSPDVEECAPNTGQCHLCLPPSEPWDQTLGVVNFPRKASRKPPREESSATCVLEQPGALGAQILLVQRPNSGLLAGLWEFPSVTWEPSEQLQRKALLQELQRWAGPLPATHLRHLGEVVHTFSHIKLTYQVYGLALEGQTPVTTVPPGARWLTQEEFHTAAVSTAMKKVFRVYQGQQPGTCMGSKRSQVSSPCSRKKPRMGQQVLDNFFRSHISTDAHSLNSAAQ

SEQ ID NO: 6 - protein, 521 aa, Artificial Sequence
  1-Isoform Gamma-3
MRKPRAAVGSGHRKQAASQEGRQKHAKNNSQAKPSACDGLARQPEEVVLQASVSSYHLFRDVAEVTAFRGSLLSWYDQEKRDLPWRRRAEDEMDLDRRAYAVWVSEVMLQQTQVATVINYYTGWMQKWPTLQDLASASLEEVNQLWAGLGYYSRGRRLQEGARKVVEELGGHMPRTAETLQQLLPGVGRYTAGAIASIAFGQATGVVDGNVARVLCRVRAIGADPSSTLVSQQLWGLAQQLVDPARPGDFNQAAMELGATVCTPQRPLCSQCPVESLCRARQRVEQEQLLASGSLSGSPDVEECAPNTGQCHLCLPPSEPWDQTLGVVNFPRKASRKPPREESSATCVLEQPGALGAQILLVQRPNSGLLAGLWEFPSVTWEPSEQLQRKALLQELQRWAGPLPATHLRHLGEVVHTFSHIKLTYQVYGLALEGQTPVTTVPPGARWLTQEEFHTAAVSTAMKKVFRVYQGQQPGTCMGSKRSQVSSPCSRKKPRMGQQVLDNFFRSHISTDAHSLNSAAQ

SEQ ID NO: 7 - DNA, 20 bp, Artificial Sequence
  Primer SUR1
agctgagagcgaggaggatg

SEQ ID NO: 8 - DNA, 20 bp, Artificial Sequence
  Primer SUR1
cacttggccagccagtagtc

SEQ ID NO: 9 - DNA, 20 bp, Artificial Sequence
  Primer GAPDH
agaacatcatccctgcatcc

SEQ ID NO: 10 - DNA, 19 bp, Artificial Sequence
  Primer GAPDH
acattgggggtaggaacac

(19) United States
(12) Patent Application Publication
(10) Pub. No.: US 2020/0171056 A1
(43) Pub. Date: Jun. 4, 2020

(54) SULFONYLUREA COMPOUNDS IN THE TREATMENT OF DISEASE ASSOCIATED WITH UV-INDUCED DAMAGE

(71) Applicant: CeMM - Forschungszentrum für Molekulare Medizin GmbH, Wien (AT)

(72) Inventors: Joanna IOANNOU LOIZOU, Wien (AT); Abdelghani MAZOUZI, Wien (AT)

(21) Appl. No.: 16/616,311

(22) PCT Filed: May 24, 2018
(86) PCT No.: PCT/EP2018/063735

§ 371 Date: Nov. 22, 2019

(30) Foreign Application Priority Data
May 24, 2017 (EP) 17172748.0

(51) Int. Cl. A61K 31/64; G01N 33/50

(52) U.S. Cl. CPC A61K 31/64; G01N 2333/924; G01N 2800/52; G01N 33/5008

(57) ABSTRACT

The present invention relates to a sulfonylurea compound for use in the treatment and/or amelioration of a disease that is associated with UV-induced DNA damage, wherein the subject to be treated expresses enzymatically active mutY homolog (MUTYH), in wherein the sufonylurea compound preferably is acetohexamide or a derivative thereof, or glimepiride or a derivative thereof. The invention furthermore relates to pharmaceutical compositions comprising a sulfonylurea compound for use in the treatment and/or amelioration of a disease that is associated with UV-induced DNA damage. Also, a screening method for identifying a compound that treats and/or ameliorates a disease that is associated with UV-induced DNA damage in a subject that expresses enzymatically active MUTYH is provided. The invention also relates to a method for monitoring the therapeutic success during the treatment of a disease that is associated with UV-induced DNA damage in a subject and a method for identifying a subject which responds to a treatment with a sulfonylurea compound.

The present invention relates to a sulfonylurea compound for use in the treatment and/or amelioration of a disease that is associated with UV-induced DNA damage, wherein the subject to be treated expresses enzymatically active mutY homolog (MUTYH), wherein the sufonylurea compound preferably is acetohexamide or a derivative thereof, or glimepiride or a derivative thereof. The invention furthermore relates to pharmaceutical compositions comprising a sulfonylurea compound for use in the treatment and/or amelioration of a disease that is associated with UV-induced DNA damage. Also, a screening method for identifying a compound that treats and/or ameliorates a disease that is associated with UV-induced DNA damage in a subject that expresses enzymatically active MUTYH is provided. The invention also relates to a method for monitoring the therapeutic success during the treatment of a disease that is associated with UV-induced DNA damage in a subject and a method for identifying a subject which responds to a treatment with a sulfonylurea compound.

Organisms have evolved a compendium of DNA repair pathways to deal with a range of different types of DNA damage in order to maintain genomic integrity and protect against cell death and disease. Nucleotide excision repair (NER) is one of the most versatile and flexible DNA repair pathways due to its capacity to deal with a wide range of structurally distinct DNA lesions. This pathway repairs ultraviolet (UV) radiation-induced lesions that are commonly in the form of cyclobutane-pyrimidine dimers (CPDs) but also 6-4 pyrimidine-pyrimidone photoproducts (6-4PPs), and also removes other bulky adducts [Marteijn et al (2014), Nat Rev Mol Cell Biol, 15: 465-81]. CPDs form rapidly upon UV exposure, and if unrepaired lead to cytosine to thymine transition mutations, which are associated with melanoma [Lo et al (2014), Science, 346: 945-9]. NER is comprised of two major sub-pathways: transcription-coupled repair (TC-NER), which functions on transcribed strands of active genes and engages RNA polymerase II in the recognition of the DNA damage; and global genome repair (GG-NER), which repairs lesions in other regions of the genome including repressed non-coding regions and non-transcribed strands of active genes [Fousteri et al (2008), Cell Res 18: 73-84]. To date, NER is the only known DNA repair pathway that repairs UV-induced DNA damage in mammalian cells.

Sulfonylurea compounds are organic compounds that are used in medicine and agriculture. Some of them are antidiabetic drugs, because they increase insulin release from beta cells of the pancreas. Acetohexamide belongs to the group of sulfonylurea compounds and is used to treat diabetes mellitus type 2. WO 2014/164730 describes acetohexamide for use in preventing malignancies, like cancer, in patients having a genetic predisposition for such a malignancy, like cancer, whereby a predisposition involves a mutation causing a loss-of-function or reduction of function in, inter alia, MUTYH.

The importance of NER as a DNA damage repair pathway is highlighted by the fact that mutations within this pathway give rise to several diseases with diverse clinical manifestations, including Xeroderma pigmentosum (XP), Cockayne syndrome (CS), UV-sensitive syndrome (UVSS) and Trichothiodystrophy (TTD). All patients display enhanced sensitivity to sunlight. Specifically, XP patients are more than 1,000 times more prone to developing cutaneous basal cell carcinoma, squamous cell carcinoma or melanoma. In addition, 20% of these patients suffer from neurological symptoms typical of neurodegeneracy. At present, there are no curative therapies for NER-deficient patients available in the art. There is just a need in the art to provide treatment options for such patients that suffer from UV-induced DNA damage.

The technical problem underlying the present invention is thus the provision of compounds and/or compositions for use in the treatment and/or amelioration of diseases associated with UV-induced DNA damage in NER-deficient subjects.

The technical problem is solved by provision of the embodiments characterized in the claims. The invention thus relates to the following items:
1. A sulfonylurea compound for use in the treatment and/or amelioration of a disease that is associated with UV-induced DNA damage, wherein the subject to be treated expresses enzymatically active mutY homolog (MUTYH), and wherein the sulfonylurea compound has the structure of Formula I:

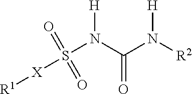

wherein
X is phenylene which is optionally substituted with —NH₂,
R¹ is selected from —C₁₋₆ alkyl, —C(O)—C₁₋₆ alkyl, —(C₁₋₆ alkylene)-C(O)—NH—R³ and —(C₁₋₆ alkylene)-NH—C(O)—R³,
wherein R³ are independently selected from monocyclic unsaturated heterocyclyl containing from 1 to 3 nitrogen atoms and optionally one or two additional heteroatoms selected from S and O, wherein the heterocyclyl optionally has one or two substituents selected from oxo (═O), -halogen, —C₁₋₆ alkyl and —O—C₁₋₆ alkyl; and
R² is selected from C₅₋₇ cycloalkyl which is optionally substituted with one or two independently selected from C₁₋₆ alkyl.
2. A pharmaceutical composition for use in the treatment and/or amelioration of a disease that is associated with UV-induced DNA damage, wherein the subject to be treated expresses enzymatically active MUTYH, and wherein the pharmaceutical composition comprises
   (i) the sulfonylurea compound for the use according to item 1; and
   (ii) optionally a pharmaceutically acceptable carrier.
3. The sulfonylurea compound for the use according to item 1, or the pharmaceutical composition for the use according to item 2, wherein the sulfonylurea compound is
   (i) acetohexamide or a derivative thereof; or
   (ii) glimepiride or a derivative thereof.
4. The sulfonylurea compound for the use according to item 1 or 3, or the pharmaceutical composition for the use according to item 2 or 3, wherein enzymatically active MUTYH is wild type MUTYH or MUTYH with increased activity.
5. The sulfonylurea compound for the use according to any one of items 1, 3 and 4, or the pharmaceutical composition for the use according to any one of items 2-4, wherein the enzymatically active MUTYH is a polypeptide comprising or consisting of
   (i) the amino acid sequence of any one of SEQ ID NOs: 1 to 6;
   (ii) an amino acid sequence having at least 80% identity to an amino acid sequence of (i), wherein the polypeptide has DNA glycosylase activity;
   (iii) the amino acid sequence of an enzymatically active fragment of SEQ ID NO: 1; or
   (iv) an amino acid sequence having at least 80% identity to an amino acid sequence of (iii), wherein the polypeptide has DNA glycosylase activity.
6. The sulfonylurea compound for the use according to any one of items 1 and 3-5, or the pharmaceutical composition for the use according to any one of items 2-5, wherein the activity of the enzymatically active MUTYH is at least 80% of the activity of a polypeptide consisting of the amino acid sequence of SEQ ID NO: 1.
7. The sulfonylurea compound for the use according to any one of items 1 and 3-6, or the pharmaceutical composition for the use according to any one of items 2-6, wherein in a sample obtained from the subject the expression amount of MUTYH is at least 80% of the expression amount of MUTYH in a sample obtained from a healthy reference subject.
8. The sulfonylurea compound for the use according to any one of items 1 and 3-7, or the pharmaceutical composition for the use according to any one of items 2-7, wherein the sample is a sample of the skin.
9. The sulfonylurea compound for the use according to any one of items 1 and 3-8, or the pharmaceutical composition for the use according to any one of items 2-8, wherein the sulfonylurea compound decreases the amount of enzymatically active MUTYH.
10. The sulfonylurea compound for the use according to any one of items 1 and 3-9, or the pharmaceutical composition for the use according to any one of items 2-9, wherein the sulfonylurea compound inhibits the enzymatic activity of MUTYH, and/or leads to the degradation and/or depletion of MUTYH.
11, The sulfonylurea compound for the use according to any one of items 1 and 3-10, or the pharmaceutical composition for the use according to any one of items 2-10, wherein the sulfonylurea compound targets MUTYH directly or indirectly via factors that mediate the inhibition of the enzymatic activity of MUTYH.
12. The sulfonylurea compound for the use according to any one of items 1 and 3-11, or the pharmaceutical composition for the use according to any one of items 2-11, wherein the sulfonylurea compound decreases the protein level of enzymatically active MUTYH in a proteasome dependent manner.
13. The sulfonylurea compound for the use according to any one of items 1 and 3-12, or the pharmaceutical composition for the use according to any one of items 2-12, wherein the sulfonylurea compound enhances the repair of UV-induced DNA damage.
14. The sulfonylurea compound for the use according item 13, or the pharmaceutical composition for the use according item 13, wherein the UV-induced DNA damage are cyclobutane-pyrimidine dimers (CPDs), 6-4 pyrimidine-pyrimidone photoproducts (6-4PPs), Dewar valence isomers and/or Spore photoproducts and other types of UV lesions.
15. The sulfonylurea compound for the use according to any one of items 1 and 3-14, or the pharmaceutical composition for the use according to any one of items 2-14, wherein the UV-induced DNA damage is caused by UVA, UVB and/or UVC irradiation.
16. The sulfonylurea compound for the use according to any one of items 1 and 3-15, or the pharmaceutical composition for the use according to any one of items 2-15, wherein the sulfonylurea compound alleviates nucleotide excision repair (NER) deficiency and/or enhances NER.
17, The sulfonylurea compound for the use according to item 16, or the pharmaceutical composition for the use according to item 16, wherein the NER is transcription-coupled repair (TC-NER) and/or global genome repair (GG-NER).
18. The sulfonylurea compound for the use according to any one of items 1 and 3-17, or the pharmaceutical composition for the use according to any one of items 2-17, wherein the disease that is associated with UV-induced DNA damage is a disease that is associated with NER deficiency.
19. The sulfonylurea compound for the use according to item 18, or the pharmaceutical composition for the use according to item 18, wherein the disease that is associated with NER deficiency is Xeroderma pigmentosum (XP), Cockayne syndrome (CS), UV-sensitive syndrome (UVSS), Trichothiodystrophy (TTD) or cerebro-oculo-facioskeletal syndrome (COFS).
20. The sulfonylurea compound for the use according to any one of items 1 and 3-19, or the pharmaceutical composition for the use according to any one of items 2-19, wherein the sulfonylurea compound alleviates symptoms associated with NER deficiency.
21. The sulfonylurea compound for the use according to item 20, or the pharmaceutical composition for the use according to item 20, wherein the symptoms associated with NER deficiency are UV sensitivity, UV-irritation, UV-induced DNA damage, UV-induced cell death, the development of cancer, neurological symptoms, premature ageing, and/or developmental defects.
22. A screening method for identifying a compound that treats and/or ameliorates a disease that is associated with UV-induced DNA damage in a subject that expresses enzymatically active MUTYH, wherein the method comprises:
   (a) contacting a test compound with
      (a1) MUTYH; or
      (a2) a cell expressing MUTYH;
   (b) measuring the expression and/or activity of MUTYH in the presence and absence of said test compound; and
   (c) identifying a compound that reduces the expression and/or activity of MUTYH as a compound that treats and/or ameliorates a disease that is associated with UV-induced DNA damage in a subject that expresses enzymatically active MUTYH,
      and optionally identifying said compound as a compound that treats and/or ameliorates a disease associated with NER deficiency, wherein said disease is preferably selected from Xeroderma pigmentosum (XP), Cockayne syndrome (CS), UV-sensitive syndrome (UVSS), Trichothiodystrophy (TTD) and cerebro-oculo-facioskeletal syndrome (COFS).
23. The screening method of item 22, wherein the activity that is measured in step (b) is DNA glycosylase activity.
24. The screening method of item 22 or 23, wherein the amount that is measured in step (b) is the amount of the MUTYH polypeptide.
25. The screening method of any one of items 22-24, wherein said cell is a eukaryotic cell.
26. The screening method of any one of items 22-25, which additionally comprises the step of
   (b2) comparing the test compound to a control.
27. The screening method of item 26, wherein in said control an inactive test compound is used, wherein said inactive test compound is a compound that does not reduce the expression and/or activity of MUTYH.
28. The screening method of any one of items 22-27, wherein said test compound is (i) a small molecule of a screening library; or
   (ii) a peptide of a phage display library, of an antibody fragment library, or derived from a cDNA library.
29. A method for monitoring the therapeutic success during the treatment of a disease that is associated with UV-induced DNA damage in a subject, wherein the method comprises:
   (a) measuring in a sample obtained from a test subject the amount and/or activity of MUTYH;
   (b) comparing said amount and/or activity with reference data corresponding to the amount and/or activity of MUTYH of at least one reference subject; and
   (c) predicting therapeutic success based on the comparison step (b).
30. The monitoring method of item 29, wherein the amount of the enzymatically active MUTYH polypeptide is measured.
31. The monitoring method of item 29 or 30, wherein the test subject has expressed enzymatically active MUTYH before the treatment started.
32. The monitoring method of item 30 or 31, wherein in a sample which was obtained from the test subject before the treatment started, the amount of the enzymatically active MUTYH polypeptide is at least 80% of the amount of the enzymatically active MUTYH polypeptide of a sample obtained from a healthy reference subject.
33. The monitoring method of any one of items 29-32, wherein the test subject is a human being who receives medication for a disease that is associated with NER deficiency.
34. The monitoring method of any one of items 29-33, wherein the reference data corresponds to the amount and/or activity of MUTYH in a sample of at least one reference subject.
35. The monitoring method of any one of items 29-34, wherein the at least one reference subject has a disease that is associated with NER deficiency but did not receive medication for this disease; and wherein in step (c) a decreased amount and/or activity of MUTYH of the test subject as compared to the reference data indicates therapeutic success in the treatment of a disease that is associated with NER deficiency.
36. The monitoring method of item 35, wherein said decreased amount and/or activity of MUTYH means that the amount and/or activity of MUTYH in the sample of the test subject is 0 to 90% of the amount and/or activity of MUTYH in the sample of the at least one reference subject.
37. The monitoring method of any one of items 29-34, wherein the at least one reference subject has a disease that is associated with NER deficiency and has received medication for this disease; and wherein in step (c) an identical or similar amount and/or activity of MUTYH of the test subject as compared to the reference data indicates therapeutic success in the treatment of a disease that is associated with NER deficiency.
38. The monitoring method of any one of items 29-34, wherein the at least one reference subject does not have a disease that is associated with NER deficiency; and wherein in step (c) an identical or similar amount and/or activity of MUTYH of the test subject as compared to the reference data indicates therapeutic success in the treatment of a disease that is associated with NER deficiency.
39. The monitoring method of item 37 or 38, wherein said identical or similar amount and/or activity of MUTYH means that the amount and/or activity of MUTYH in the sample of the test subject is 90-110% of the amount and/or activity of MUTYH in the sample of the at least one reference subject.

A method for identifying a subject which responds to a treatment with a sulfonylurea compound as defined in any one of items 1-21, wherein the method comprises:
(a) measuring the expression and/or activity of MUTYH in a sample obtained from a test subject; and
(c) identifying a subject which comprises enzymatically active MUTYH as a responder to a treatment with a sulfonylurea compound as defined in any one of items 1-21.
41. The method of item 40, wherein the subject has a disease that is associated with UV-induced DNA damage.
42. The method of item 40 or 41, wherein the amount of enzymatically active MUTYH in the sample of the test subject is at least as high has the amount of enzymatically active MUTYH of a sample of a healthy reference subject.
43. A method for ameliorating/treating a disease a disease that is associated with UV-induced DNA damage, wherein the subject to be treated expresses enzymatically active mutY homolog (MUTYH), wherein the method comprises administering to the subject a sulfonylurea compound having the structure of Formula I:

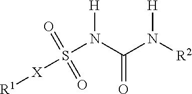

wherein
X is phenylene which is optionally substituted with —NH₂,
R¹ is selected from —C₁₋₆ alkyl, —C(O)—C₁₋₆ alkyl, —(C₁₋₆ alkylene)-C(O)—NH—R³ and —(C₁₋₆ alkylene)-NH—C(O)—R³,
wherein R³ are independently selected from monocyclic unsaturated heterocyclyl containing from 1 to 3 nitrogen atoms and optionally one or two additional heteroatoms selected from S and O, wherein the heterocyclyl optionally has one or two substituents selected from oxo (═O), -halogen, —C₁₋₆ alkyl and —O—C₁₋₆ alkyl; and
R² is selected from C₅₋₇ cycloalkyl which is optionally substituted with one or two independently selected from C₁₋₆ alkyl.

Accordingly, the present invention relates to sulfonylurea compounds for use in the treatment and/or amelioration of diseases that are associated with UV-induced DNA damage, wherein the subject to be treated expresses enzymatically active mutY homolog (MUTYH), and wherein the sulfonylurea compound has the structure of Formula I:

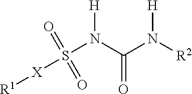

wherein
X is phenylene which is optionally substituted with —NH₂,
R¹ is selected from —C₁₋₆ alkyl, —C(O)—C₁₋₆ alkyl, —(C₁₋₆ alkylene)-C(O)—NH—R³ and —(C₁₋₆ alkylene)-NH—C(O)—R³,
wherein R³ are independently selected from monocyclic unsaturated heterocyclyl containing from 1 to 3 nitrogen atoms and optionally one or two additional heteroatoms selected from S and O, wherein the heterocyclyl optionally has one or two substituents selected from oxo (═O), -halogen, —C₁₋₆ alkyl and —O—C₁₋₆ alkyl; and
R² is selected from C₅₋₇ cycloalkyl which is optionally substituted with one or two independently selected from C₁₋₆ alkyl.
In a preferred embodiment of the invention, X in Formula I is unsubstituted phenylene.
In another preferred embodiment of the invention, R¹ of Formula I is selected from -methyl, —C(O)-methyl, —(C₁₋₃ alkylene)-C(O)—NH—R³ and —(C₁₋₃ alkylene)-NH—C(O)—R³,

In a preferred embodiment, the invention relates to the sulfonylurea compounds of formula I, wherein R³ are independently selected from 5 or 6 membered monocyclic unsaturated heterocyclyl containing 1 nitrogen atom, wherein the heterocyclyl optionally has one or two substituents selected from oxo (═O), -halogen, —C₁₋₃ alkyl and —O-methyl.

The invention also relates to the sulfonylurea compound for use according to the invention, wherein R² of Formula I is selected from cyclohexyl which is optionally substituted with methyl.

As used herein, the term “alkyl” refers to a monovalent saturated acyclic (i.e., non-cyclic) hydrocarbon group which may be linear or branched. Accordingly, an “alkyl” group does not comprise any carbon-to-carbon double bond or any carbon-to-carbon triple bond. A “C₁₋₆ alkyl” denotes an alkyl group having 1 to 6 carbon atoms. Preferred exemplary alkyl groups are methyl, ethyl, propyl (e.g., n-propyl or isopropyl), or butyl (e.g., n-butyl, isobutyl, sec-butyl, or tert-butyl). Unless defined otherwise, the term “alkyl” preferably refers to C₁₋₃ alkyl, more preferably to methyl or ethyl, and even more preferably to methyl.

As used herein, the term “alkylene” refers to an alkanediyl group, i.e. a divalent saturated acyclic hydrocarbon group which may be linear or branched. A “C₁₋₆ alkylene” denotes an alkylene group having 1 to 6 carbon atoms. Preferred exemplary alkylene groups are methylene (—CH₂—), ethylene (e.g., —CH₂—CH₂— or —CH(—CH₃)—), propylene (e.g., —CH₂—CH₂—CH₂—, —CH(—CH₂—CH₃)—, —CH₂—CH(—CH₃)—, or —CH(—CH₃)—CH₂—), or butylene (e.g., —CH₂—CH₂—CH₂—CH₂—). Unless defined otherwise, the term “alkylene” preferably refers to C₁₋₃ alkylene (including, in particular, linear C₁₋₃ alkylene), more preferably to methylene or ethylene, and even more preferably to ethylene.

As used herein, the term “unsaturated heterocyclyl” refers to a ring group, wherein said ring group comprises one or more (such as, e.g., one, two, three, or four) ring heteroatoms independently selected from O, S and N, and the remaining ring atoms are carbon atoms, wherein one or more S ring atoms (if present) and/or one or more N ring atoms (if present) may optionally be oxidized, wherein one or more carbon ring atoms may optionally be oxidized (i.e., to form an oxo group), and further wherein said ring group may be partially unsaturated (i.e., unsaturated but not aromatic) or aromatic. Examples of the “monocyclic unsaturated heterocyclyl” include to pyrrolyl (e.g., 2H pyrrolyl), pyrrolone (e.g. (5H)-pyrrol-2-one), imidazolyl, pyrazolyl, pyridyl (i.e., pyridinyl; e.g., 2 pyridyl, 3 pyridyl, or 4 pyridyl), pyrazinyl, pyrimidinyl, pyridazinyl, thiazolyl, isothiazolyl, oxazolyl, isoxazolyl, furazanyl. Preferred examples include pyrrolones and pyridine. It is to be understood that in the monocyclic unsaturated heterocyclyl containing from 1 to 3 nitrogen atoms and optionally one or two additional heteroatoms selected from S and O, the remaining ring members which are not N, S or O, are carbon atoms. The number of carbon atoms is preferably from 3 to 5. Likewise, in the 5 or 6 membered monocyclic unsaturated heterocyclyl containing 1 nitrogen atom, the ring members other than the nitrogen atom are carbon atoms. In this case, the number of carbon atoms is preferably 4 or 5.

As used herein, the term “cycloalkyl” refers to a saturated hydrocarbon ring group, including monocyclic rings as well as bridged ring, Spiro ring and/or fused ring systems (which may be composed, e.g., of two or three rings; such as, e.g., a fused ring system composed of two or three fused rings). “Cycloalkyl” may, e.g., refer to cyclopropyl, cyclobutyl, cyclopentyl, cyclohexyl, cycloheptyl, or adamantyl. Unless defined otherwise, “cycloalkyl” preferably refers to a C₃₋₁₁ cycloalkyl, and more preferably refers to a C₃₋₇ cycloalkyl. A particularly preferred “cycloalkyl” is a monocyclic saturated hydrocarbon ring having 3 to 7 ring members. Most preferably, the term “cycloalkyl” refers to a cyclohexyl group.

As used herein, the term “halogen” refers to fluoro (—F), chloro (—Cl), bromo (—Br), or iodo (—I).

As used herein, the terms “optional”, “optionally” and “may” denote that the indicated feature may be present but can also be absent. Whenever the term “optional”, “optionally” or “may” is used, the present invention specifically relates to both possibilities, i.e., that the corresponding feature is present or, alternatively, that the corresponding feature is absent. For example, the expression “X is optionally substituted with Y” (or “X may be substituted with Y”) means that X is either substituted with Y or is unsubstituted. Likewise, if a component of a composition is indicated to be “optional”, the invention specifically relates to both possibilities, i.e., that the corresponding component is present (contained in the composition) or that the corresponding component is absent from the composition.

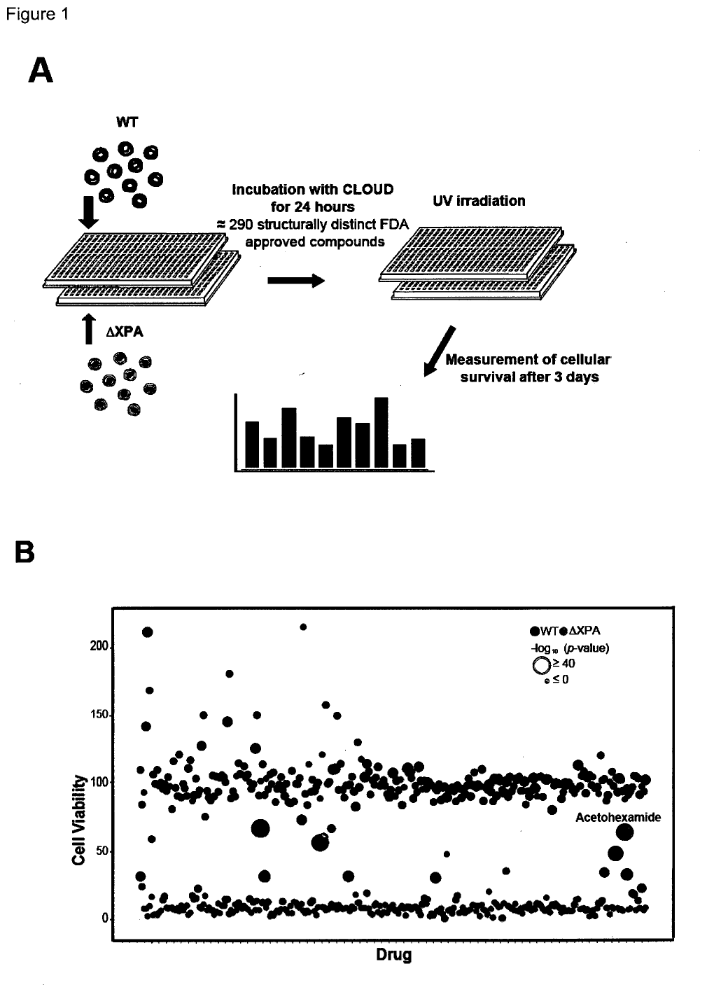

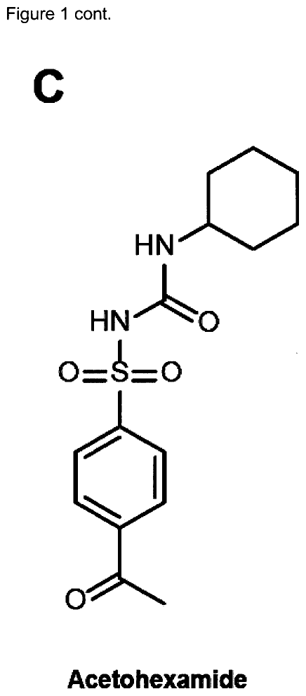

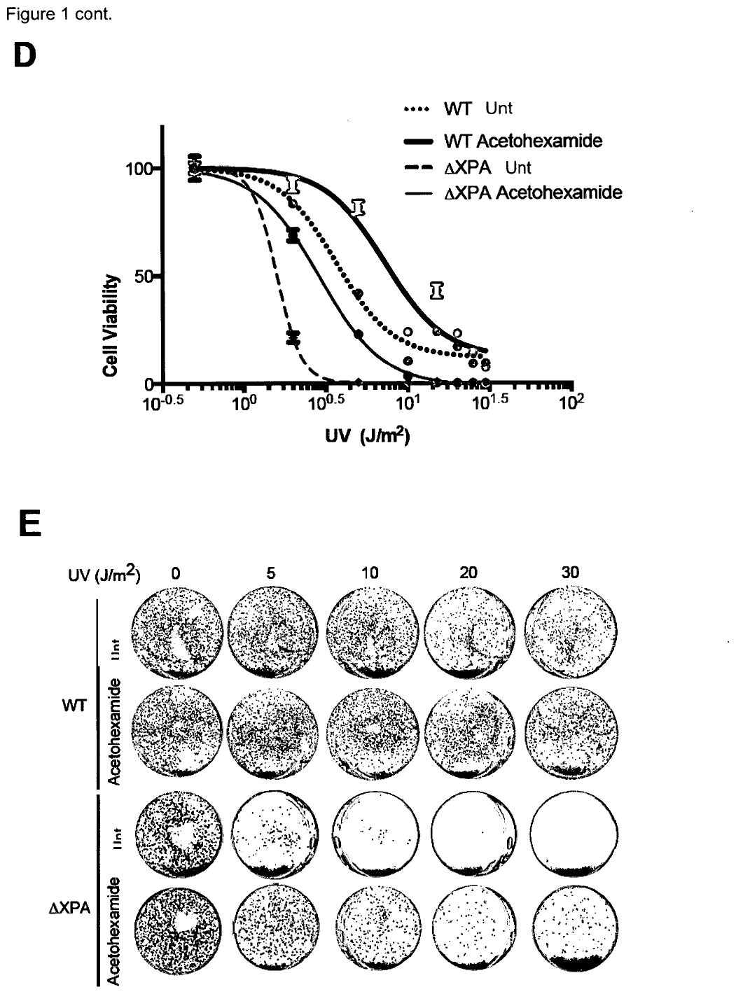

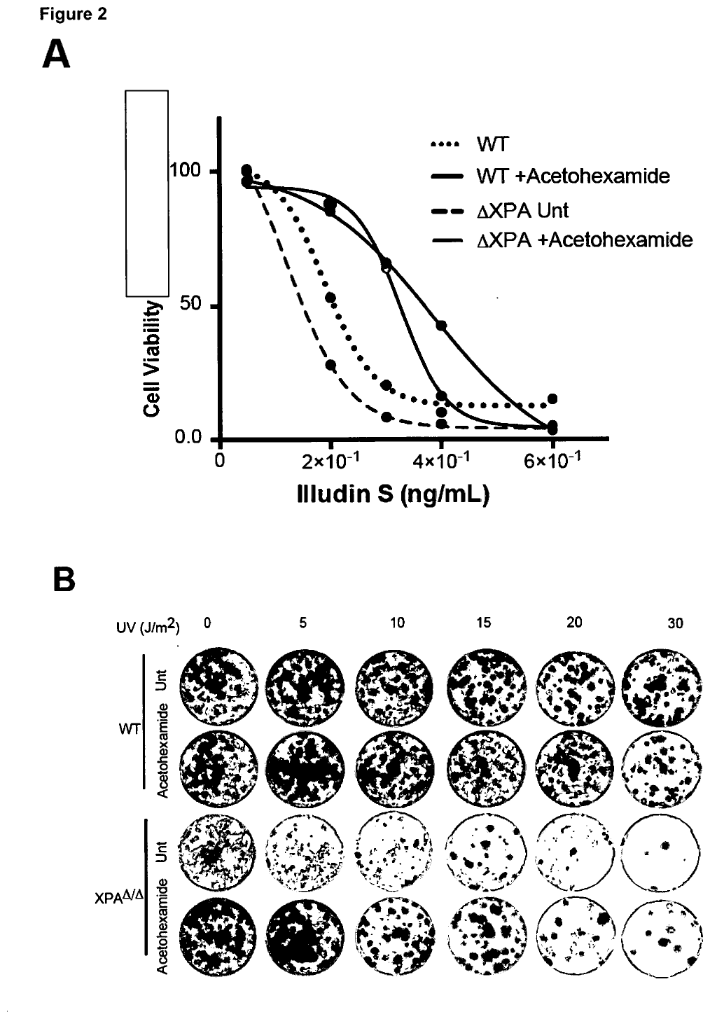

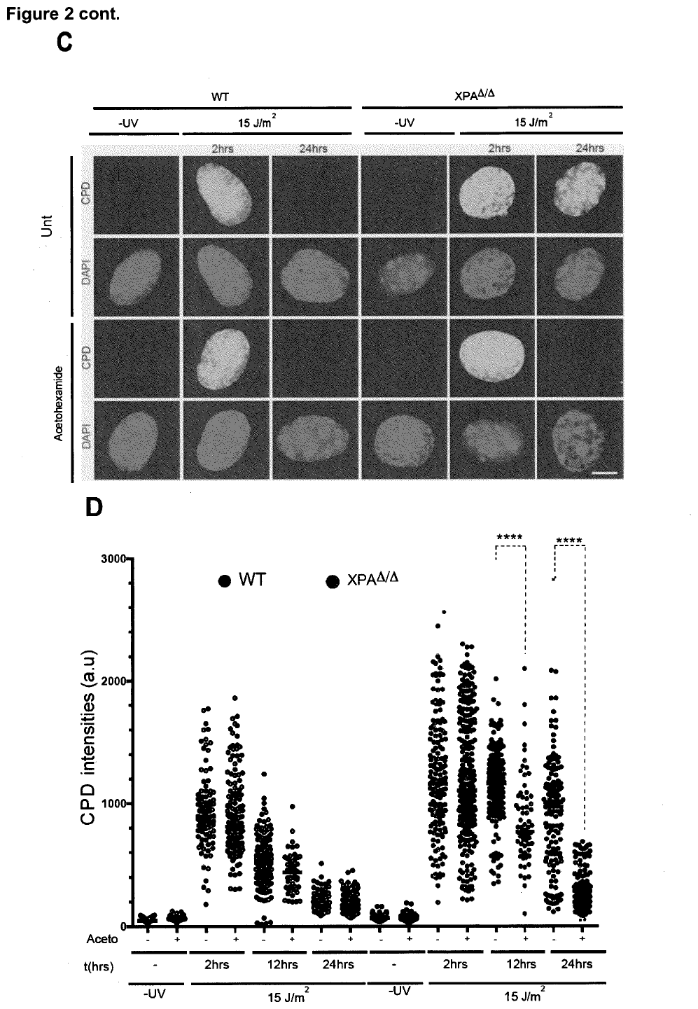

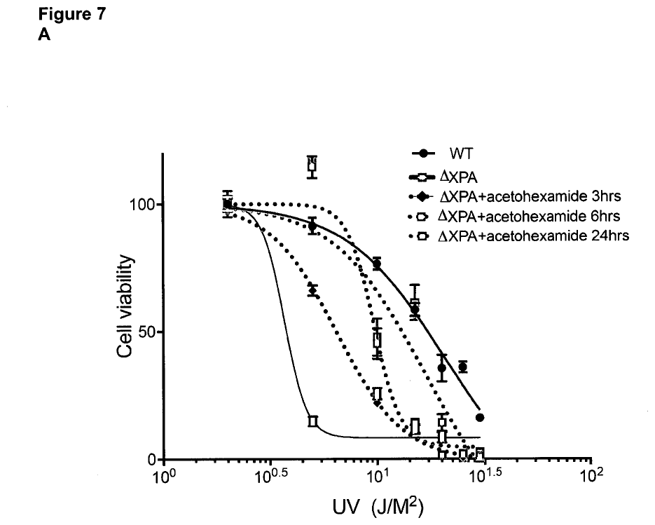

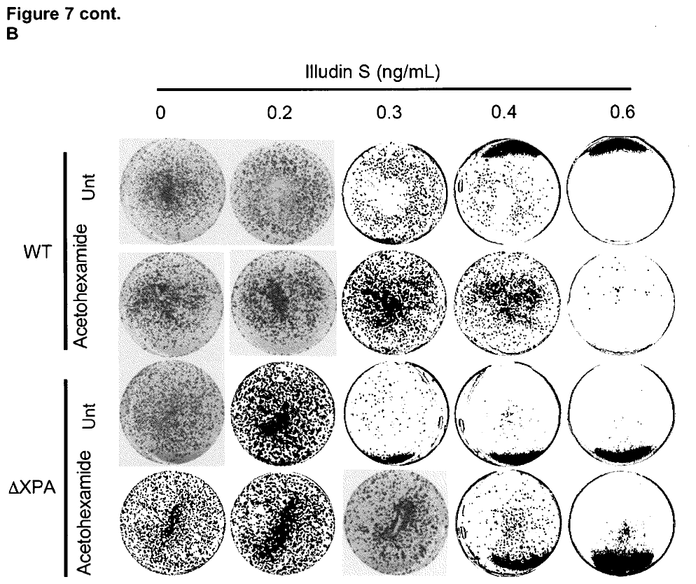

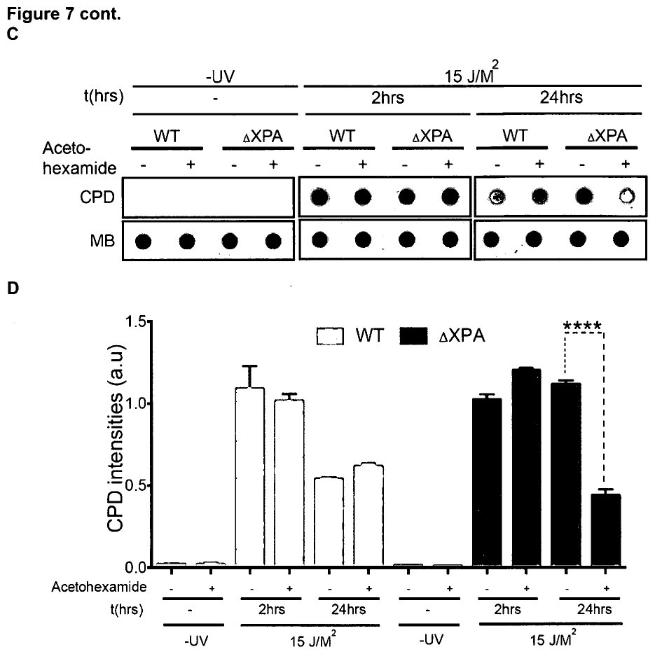

It was thus surprisingly found by the inventors that sulfonylurea compounds of the invention, in particular acetohexamide or derivatives thereof or glimepiride or derivates thereof, alleviate the UV sensitivity of cells almost to the level of wildtype cells both in a short-term dose response assay (FIG. 1D) and in a long-term colony formation assay (FIG. 1E). In this respect, the inventors determined whether incubation with sulfonylurea compounds of the invention, in particular acetohexamide or derivatives thereof or glimepiride or derivates thereof, leads to a clearance of UV-induced lesions by measuring the levels of CPDs, the most predominant lesions induced by UV and representing approximately 75% of UV lesions. As expected wildtype cells that are NER proficient were able to dear CPDs 24 hours post UV irradiation, whereas, NER-deficient XPA^{Δ/Δ} cells continued to show elevated levels of CPDs at 24 hours post UV irradiation. Very surprisingly and entirely unexpectedly, sulfonylurea compounds of the invention, in particular acetohexamide or derivatives thereof or glimepiride or derivates thereof, led to the clearance of CPDs in XPA^{Δ/Δ} cells, proving that the compounds of the invention, in particular acetohexamide or derivatives thereof or glimepiride or derivates thereof, enhance the ability of NER-deficient cells to clear CPDs lesions. Importantly, the initial amount of CPDs was not affected (FIGS. 2C-D). The same surprising observation was also made for HAP1 cell lines (FIGS. 7C-D). The sulfonylurea compounds of the invention, in particular acetohexamide or derivatives thereof or glimepiride or derivates thereof, additionally or alternatively enhance the ability of NER-deficient cells to clear pyrimidine (6-4) pyrimidone photoproducts (6-4 PPs). 6-4PPS are lesions characterized by a covalent bond that links the C6 of the 5′-end to the C4 of the 3′-end pyrimidine while the C4 exocyclic group of the original 3′-end base is shifted to the C5 position of the 5′-end pyrimidine.

This surprising finding opens up new therapeutic approaches for the treatment of the many NER-associated diseases, including but not limited to Xeroderma pigmentosum (XP), Cockayne syndrome (CS), UV-sensitive syndrome (UVSS) and Trichothiodystrophy (TTD).

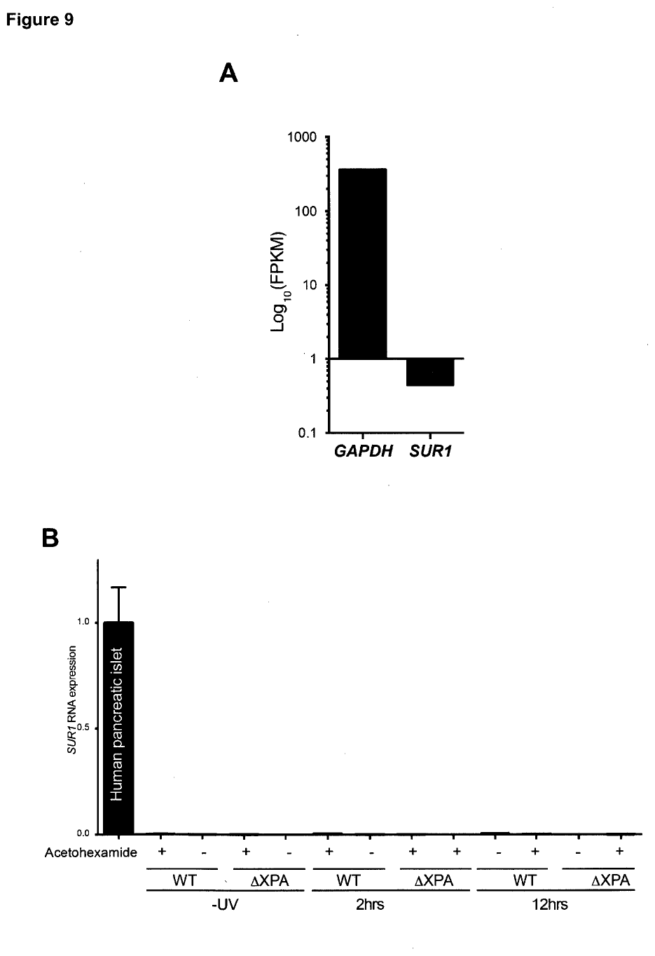

To gain insight into the mode of action of the compounds of the invention, in particular acetohexamide or derivatives thereof or glimepiride or derivates thereof, cell cycle profiles were assessed upon exposure to the compound. There was no difference between wildtype or ΔXPA cells upon treatment, ruling out an effect on cell cycle phase (FIG. 9A). To exclude the possibility that the compounds of the invention, in particular acetohexamide or derivatives thereof or glimepiride or derivates thereof, have a general anti-apoptotic effect, wildtype cells were treated with a variety of different DNA damaging agents including the DNA crosslinking agent mitomycin C (MMC), hydroxyurea (HU), which depletes cellular pools of ribonucleosides thereby inducing replication stress, and the alkylating agent methyl methanesulfonate (MMS). The compounds of the invention, in particular acetohexamide or derivatives thereof or glimepiride or derivates thereof, did not increase cellular survival following exposure to MMC, HU and MMS. Thus, the compounds of the invention, in particular acetohexamide or derivatives thereof or glimepiride or derivates thereof, do not act as an anti-apoptotic agent following DNA damage (FIG. 9B-D). Furthermore, the potent antioxidant N-acetylcysteine (NAC) showed a very minor effect in alleviating UV-induced sensitivity compared to acetohexamide (FIG. 9E), suggesting that the compounds of the invention, in particular acetohexamide or derivatives thereof or glimepiride or derivates thereof, are not exerting their effect simply by quenching reactive oxygen species.

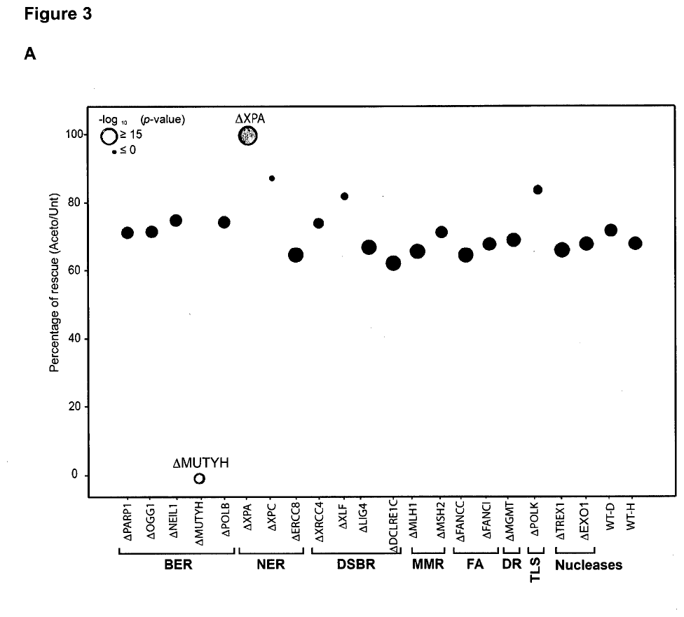

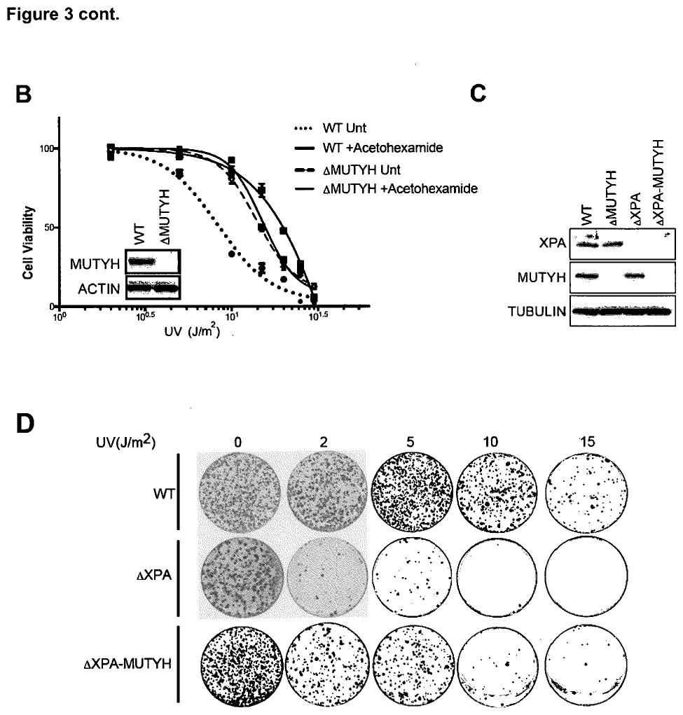

Since the sulfonylurea compounds of the invention, in particular acetohexamide or derivatives thereof or glimepiride or derivates thereof, enhanced the clearance of CPDs in NER-deficient cells, a panel of 20 DNA repair-deficient cell lines using CRISPR-Cas9 was generated, representing all DNA repair pathways. Pol kappa (POLK) was selected to represent translesion synthesis (TLS) polymerases since it has roles in the repair synthesis step of NER. Subsequently, these cell lines were treated (as well as two wildtype controls) with the compounds of the invention, in particular acetohexamide or derivatives thereof or glimepiride or derivates thereof, and exposed to UV irradiation (FIG. 3A). The ‘percentage of rescue’ was defined as the difference in survival of a given cell line treated with acetohexamide compared to untreated, following UV irradiation (FIG. 3A). Surprisingly, the sunfonylurea compounds of the invention, in particular acetohexamide or derivatives thereof or glimepiride or derivates thereof, had a comparable protective effect against UV-induced damage on all the knockout cell lines tested (and also to wildtype cells) but had no effect on cells lacking MUTYH. These results surprisingly and unexpectedly demonstrate that sulfonylurea compounds of the invention, such as acetohexamide or derivatives thereof or glimepiride or derivates thereof, and MUTYH have a related function and that the compounds of the invention have a general effect on protecting cells against UV-induced DNA damage.

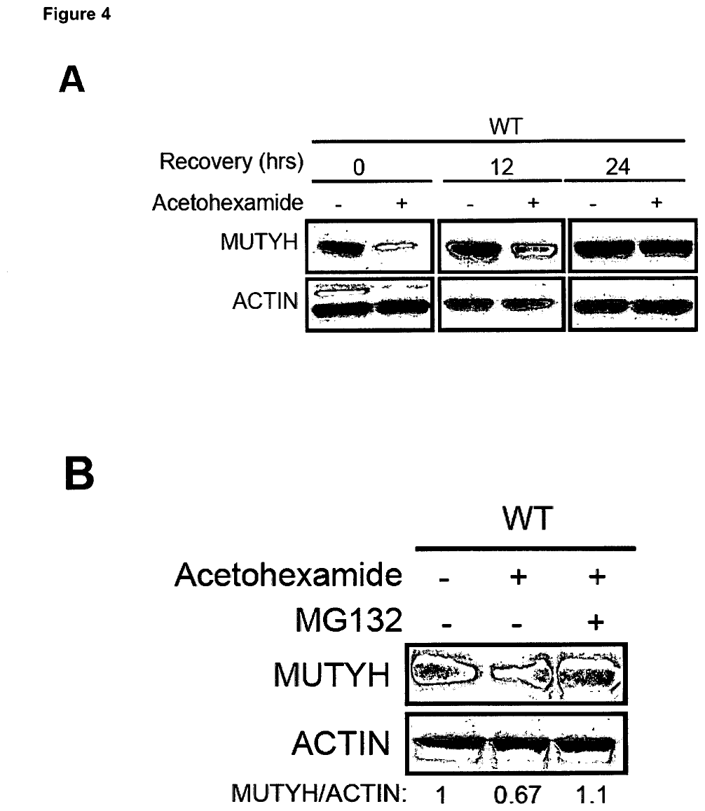

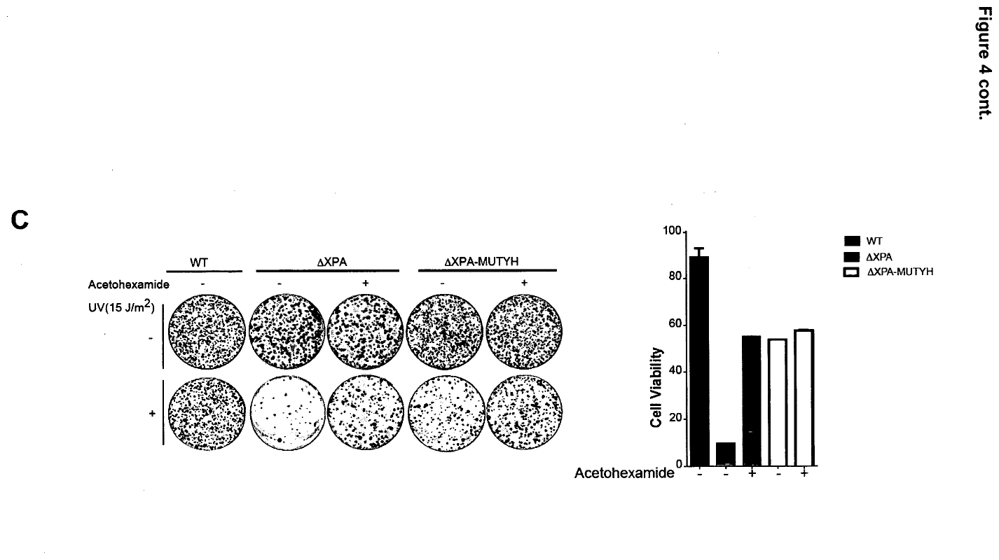

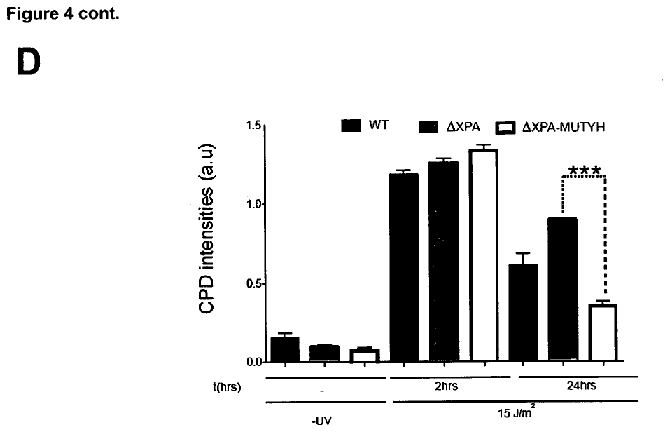

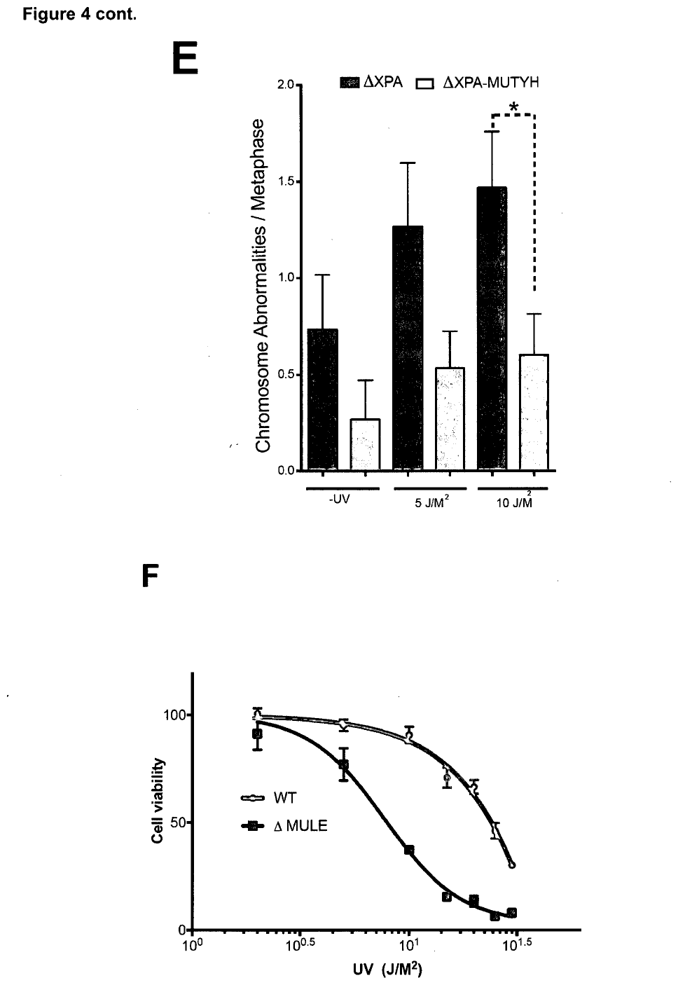

In this regard, MUTYH is a DNA glycosylase that catalyzes the excision of the adenine mis-paired with 8-oxo-guanine in the base excision repair (BER) pathway. Thus, MUTYH is an unusual glycosylase since it removes an undamaged base situated opposite a DNA lesion, instead of removing the damaged base [Markkanen et al (2013), Front Genet, 4: 18]. It was found that loss of MUTYH conferred resistance to UV irradiation compared to wildtype cells, similar to the effect of treatment using the compounds of the invention. Furthermore, pre-incubation with acetohexamide did not have a noticeable effect on survival (FIG. 3B), further suggesting that acetohexamide and loss of MUTYH have functionally related effects. It was furthermore determined whether acetohexamide works via MUTYH. Thus, the effect on MUTYH protein levels was analysed. Surprisingly, it was found that treatment using the compounds of the invention of wildtype cells led to a decrease in MUTYH protein levels in a proteasome dependent manner (FIG. 4A-B). Accordingly, the sulfonylurea compounds of the invention, in particular acetohexamide or derivatives thereof or glimepiride or derivates thereof, exhibit their functions by promoting the degradation of MUTYH. In further support of this, acetohexamide treatment of the double knockout ΔXPA-MUTYH did not lead to a further increase in survival upon UV treatment (FIG. 4C).

Thus, the inventors have surprisingly and unexpectedly found that sulfonylurea compounds of the invention, in particular acetohexamide or derivatives thereof or glimepiride or derivates thereof, alleviate the sensitivity of NER-deficient cells and enhance the repair of UV lesions through degradation of MUTYH. Thus, the invention relates to a sulfonylurea compound for use in the treatment and/or amelioration of a disease that is associated with UV-induced DNA damage, wherein the subject to be treated expresses enzymatically active mutY homolog (MUTYH), and wherein the sulfonylurea compound has the structure of formula I.

In a particular embodiment, the sulfonylurea compound of the invention is acetohexamide or a derivate thereof. In another particular embodiment of the invention, the sulfonylurea compound of the invention is glimepiride or a derivate thereof.

In the present invention, the subject to be treated expresses enzymatically active mutY homolog (MUTYH). In one embodiment of the invention, enzymatically active MUTYH is wild type MUTYH or MUTYH with increased activity. The person skilled in the art knows the MUTYH enzyme and is well-aware that its amino acid sequence as well as nucleotide sequence and/or sequences of isoforms thereof can be found in known databases such as GenBank. In a preferred embodiment of the invention, however, MUTYH is a polypeptide comprising or consisting of the amino acid sequence of MUTYH isoforms alpha-1, alpha-2, alpha-3, beta-1, gamma-2 or gamma-3. Accordingly, MUTYH is preferably a polypeptide comprising or consisting of the amino acid sequence of any one of SEQ ID NOs 1, 2, 3, 4, 5 or 6 or a polypeptide having at least 80, 85, 90, 91, 92, 93, 94, 95, 96, 97, 98, or 99% identity to an amino acid sequence of any one of SEQ ID NOs 1, 2, 3, 4, 5 or 6, wherein the polypeptide has an activity corresponding to the activity of wild-type MUTYH. In an in vivo context, MUTYH has DNA glycosylase activity. The DNA glycosylase activity of the enzymatically active MUTYH can be tested by incubating a purified polypeptide supposed to be enzymatically active MUTYH, for example by determining its amino acid sequence and finding that it has at least 80, 85, 90, 91, 92, 93, 94, 95, 96, 97, 98, or 99% identity to an amino acid sequence of any one of SEQ ID NOs 1, 2, 3, 4, 5 or 6, with a radiolabeled oligo, containing DNA lesions, including guanine, 8-oxo-7,8-dihydroguanine or 2-hydroxy-adenine, CPDs and 6-4PPs, and measuring cleavage activity in the complementary non-damaged strand. If cleavage activity is determined, the tested polypeptide is determined to be enzymatically active MUTYH within the meaning of the present invention. In one embodiment, the enzymatically active MUTYH has the amino acid sequence of an enzymatically active fragment of any one of SEQ ID NOs: 1, 2, 3, 4, 5 or 6, wherein preferred fragments comprise one or more, preferably all, of the ENDO3c, endonuclease III fragment comprising amino acids 125-283 of any one of SEQ ID NOs 1, 2, 3, 4, 5 or 6, the FeS, iron-sulphur binding domain comprising amino acids 286-306 of any one of SEQ ID NOs 1, 2, 3, 4, 5 or 6, and/or the DNA glycosylase domain comprising amino acids 365-494 of any one of SEQ ID NOs 1, 2, 3, 4, 5 or 6. In one embodiment, the amino acid sequence of the enzymatically active MUTYH polypeptide has at least 80, 85, 90, 91, 92, 93, 94, 95, 96, 97, 98 or 99% identity to an amino acid sequence of an enzymatically active fragment of any one of SEQ ID NOs: 1, 2, 3, 4, 5 or 6, wherein preferred fragments comprise one or more, preferably all, of the ENDO3c, endonuclease III fragment comprising amino acids 125-283 of any one of SEQ ID NOs 1, 2, 3, 4, 5 or 6, the FeS, iron-sulphur binding domain comprising amino acids 286-306 of any one of SEQ ID NOs 1, 2, 3, 4, 5 or 6, and/or the DNA glycosylase domain comprising amino acids 365-494 of any one of SEQ ID NOs 1, 2, 3, 4, 5 or 6, wherein the polypeptide has DNA glycosylase activity. In a preferred embodiment, the DNA glycosylase activity of the enzymatically active MUTYH is tested by incubating a purified polypeptide supposed to be enzymatically active MUTYH, for example by determining its amino acid sequence, with a radiolabeled oligo, containing DNA lesions, including guanine, 8-oxo-7,8-dihydroguanine or 2-hydroxy-adenine, CPDs and 6-4PPs, and measure cleavage activity in the complementary non-damaged strand. If cleavage activity is determined, the tested polypeptide is determined to be enzymatically active MUTYH within the meaning of the present invention.

Accordingly, the term “enzymatically active MUTYH” as used herein means that the polypeptide has DNA glycosylase activity, catalyzing the excision of the adenine mis-paired with guanine, 8-oxo-7,8-dihydroguanine or 2-hydroxy-adenine. Enzymatically active MUTYH cleaves the N-glycosidic bond between the target base and its deoxyribose sugar, leaving an apurinic/apyrimidinic (AP) site. The phosphodiester bond 5′ to the AP site is then cleaved by AP endonuclease 1 (APE1), and downstream BER enzymes complete the repair process.

In a preferred embodiment of the invention, the activity of the enzymatically active MUTYH as used herein or the fragment thereof is at least 80, 85, 90, 91, 92, 93, 94, 95, 96, 97, 98, 99 or 100% of the activity of a polypeptide consisting of the amino acid sequence of SEQ ID NO: 1. The activity is preferably determined using the test described above. However, the person skilled in the art is able to set up alternative tests to determine whether a given polypeptide has MUTYH activity.

In one embodiment of the invention, in a sample obtained from the subject to receive the sulfonylurea compound of the invention, the amount of MUTYH is determined. In a preferred embodiment, the expression amount of MUTYH in the sample obtained from the subject is at least 80, 85, 90, 91, 92, 93, 94, 95, 96, 97, 98, 99 or 100% of the expression amount of MUTYH in a sample obtained from a healthy reference subject. To determine the expression amount of MUTYH, any technique suitable for that purpose and known to the person skilled in the art can be employed. For example, immunoblotting, mass spectrometry techniques, Enzyme-Linked Immunosorbent Assay (ELISA), Flow cytometry based methods (FACS), Immunohistochemistry based methods or Immunofluorescence based methods can be employed. The person skilled in the art is well-aware how an experimental set up using any of the above methods can be chosen to reliably determine the expression amount of MUTYH in a sample obtained from the subject to receive the sulfonylurea compound of the invention and/or in a sample obtained from a healthy reference subject. In a preferred embodiment of the invention, the sample obtained from the subject to receive the sulfonylurea compounds of the invention and from the healthy subject is a skin sample or a blood sample.

As detailed further above, it was unexpectedly found that treatment using the compounds of the invention of wildtype cells led to a decrease in MUTYH protein levels in a proteasome dependent manner (FIG. 4A-B). Accordingly, the compounds of the invention, in particular acetohexamide or derivatives thereof or glimepiride or derivates thereof, exhibit their functions by promoting the degradation of MUTYH. Thus, in one embodiment, the sulfonylurea compound of the invention decreases the amount of enzymatically active MUTYH and/or inhibits the enzymatic activity of MUTYH, and/or leads to the degradation and/or depletion of MUTYH. The sulfonylurea compounds of the invention target MUTYH, in one embodiment of the invention, directly or indirectly via factors that mediate the inhibition of the enzymatic activity of MUTYH. In this respect, the inventors demonstrated that the sufonylurea compounds of the invention alleviate the sensitivity of NER-deficient cells and enhance the repair of UV lesions through degradation of MUTYH. It has been shown that MUTYH is ubiquitinated by the E3 ligase MULE, thereby reducing its protein levels and subsequent recruitment to chromatin [Dorn et al (2014), J Biol Chem, 289: 7049-58]. Hence the loss of MULE sensitizes cells to UV irradiation due to an accumulation of MUTYH protein. The inventors were able to demonstrate that MULE-deficient cells (AMULE) also showed enhanced sensitivity to UV irradiation (FIG. 4F). Taken together, without being bound by theory, the sufonylurea compounds function by inhibiting a deubiquitin ligase that in turn leads to increased MUTYH ubiquitination by MULE and subsequent degradation. Hence, the sulfonylurea compounds of the invention target MUTYH, in one embodiment of the invention, directly or indirectly via factors that mediate the inhibition of the enzymatic activity of MUTYH, whereby a factor mediating the inhibition of the enzymatic activity of MUTYH and/or that may regulate MUTYH protein levels is a deubiquitin ligase that opposes the effect of Mule or other ubiquitin ligases that target MUTYH for ubiquitination and degradation. Thus, in one embodiment, the sulfonylurea compound of the invention decreases the protein level of enzymatically active MUTYH in a proteasome dependent manner.

In one embodiment, the sulfonylurea compound of the invention enhances the repair of UV-induced DNA damage.

The term “UV-induced DNA damage” as used herein refers to alterations of the DNA caused by UV light, for example UV light emitted by the Sun. Ultraviolet (UV) is an electromagnetic radiation with a wavelength from 10 nm (30 PHz) to 400 nm (750 THz), shorter than that of visible light but longer than X-rays. UV radiation constitutes about 10% of the total light output of the Sun, and is thus present in sunlight. It is also produced by electric arcs and specialized lights, such as mercury-vapor lamps, tanning lamps, and black lights. Although it is not considered an ionizing radiation because its photons lack the energy to ionize atoms, long-wavelength ultraviolet radiation can cause chemical reactions and causes many substances to glow or fluoresce. Consequently, the biological effects of UV are greater than simple heating effects, and many practical applications of UV radiation derive from its interactions with organic molecules. One effect of UV light on DNA is to cause damages, i.e. alterations of the DNA compared to its status before UV light exposure. In the present invention, UV-induced DNA damages are cyclobutane-pyrimidine dimers (CPDs), 6-4 pyrimidine-pyrimidone photoproducts (6-4PPs), Dewar valence isomers and/or Spore photoproducts and other types of UV lesions. Cyclobutane-pyrimidine dimers (CPDs) are formed by cycloaddition between the C5-C6 double bonds of the two pyrimidine moieties. This reaction gives rise to the formation of a 4 membered cyclobutane ring linking the two bases. CPDs can be formed between adjacent pyrimidines including: Thymine-Thymine (TT), Cytosine-Thymine (CT), Thymine-Cytosine (TC) and Cytosine-Cytosine. They can also be formed for 5-methylcytosine (m⁵C). Several arrangements can be adapted by the two pyrimidine bases with the respect to the cyclobutane moiety. If the bases are in the same strand as the cyclobutane ring, this geometrical structure is known as cis stereoisomers. In contrast, if the bases are in the opposite strand of the cyclobutane ring then they are defined as trans stereoisomers. More complexity can be observed with regard to the covalent bonds that are formed between the bases. If the C5 of one pyrimidine is linked to the C5 of the other pyrimidine and the C6 atoms are also linked to each other in a parallel structure, this is known as syn configuration, whereas the anti configuration occurs when the C5 binds with the C6 in an antiparallel orientation. Other photoproducts which are different from CPDs are 5-(α-thyminyl)-5,6-dihydrothymine (Spore photoproducts). Pyrimidine (6-4) pyrimidone photoproducts (6-4 PPs) are lesions characterized by a covalent bond that links the C6 of the 5′-end to the C4 of the 3′-end pyrimidine while the C4 exocyclic group of the original T-end base is shifted to the C5 position of the 5′-end pyrimidine. 6-4PPs can be also converted to another structure known as Dewar valence isomers (DEWs), which are characterized by covalent bonds between the N3 and C6 atoms of pyrimidine. Both 6-4PPs and DEWs can be deaminated when the 5′-end is a cytosine. CPDs, 6-4PPs and DEWs represent the most frequent UV-induced DNA lesions, however a few other photoproducts also exist. Dimeric photoproducts involving adenine and thymine, or two adenine rings, or the stereoisomers of 6-hydroxy-5,6-dihydrocytosine (known as cytosine hydrates) have been characterized in a model system exposed to UVC radiation. In a preferred embodiment, UV damages are those caused by UVA, UVB and/or UVC irradiation. In this respect, UVA radiation relates to radiation having a wavelength between 315-400 nm, UVB radiation relates to radiation having a wavelength between 280-315 nm and UVC radiation relates to radiation having a wavelength between 100-280 nm.

In accordance with the findings of the inventors, the compounds of the present invention, in one embodiment, alleviate nucleotide excision repair (NER) deficiency and/or enhance NER. In a preferred embodiment, the NER is transcription-coupled repair (TC-NER) and/or global genome repair (GG-NER).

As used herein, the term Nucleotide excision repair (NER) refers to a very versatile and flexible pathway for repair because it has the capacity to cope with structurally distinct DNA lesions. This pathway repairs ultraviolet (UV) radiation-induced lesions that are commonly in the form of cyclobutane-pyrimidine dimers (CPDs) but also 6-4 pyrimidine-pyrimidone photoproducts (6-4PPs), Dewar valence isomers, Spore photoproducts as well as other lesions such as intrastrand crosslinks and several other bulky adducts such as cyclopurines. There are approximately 30 proteins involved in the NER pathway, that cooperate together to ensure the appropriate and precise repair of the DNA lesion through four main basic steps: damage recognition, excision of the damaged DNA strand, DNA synthesis and DNA ligation. NER is comprised of two major sub-pathways, based on the recognition and the location of the damage in the genome: global genome repair (GG-NER) and transcription coupled repair (TC-NER) that acts on transcribed strands of active genes and engages RNA polymerase II in the recognition of the DNA damage. In this respect, Global genome repair pathway (GG-NER) deals with DNA damage throughout the genome including at repressed non-coding regions. As with many other DNA repair pathways, GG-NER is initiated by DNA damage detection and recognition. The former consists of scanning the whole genome for helix distortions and changes in the conformation and the structure of the nucleotides. The major DNA lesion detector in GG-NER is a complex that consists mainly of XPC, UV-excision repair protein RAD23 homolog B (RAD23B) and centrin 2 (CETN2). Even though XPC is the major protein in detecting UV lesions in GG-NER, CPDs are hardly recognized by XPC due to its mild thermodynamic duplex destabilization of the double helix. To deal with this type of lesions, recently, XPC was shown to be recruited to chromatin via the ultraviolet radiation-DNA damage-binding protein complex (UV-DDB-associated E3). After the damage is recognized by XPC, the transcription initiation factor IIH (TFIIH) is recruited, which is composed of ten protein subunits, including XPB and XPD. Subsequently, the damage is excised by XPF-ERCC1 and XPG endonucleases at 5′ and 3′ respectively at short distances away from the lesion, resulting in a single strand gap of 22 to 30 nucleotides. XPA is one of the central components of NER due its versatile functions, it is very important in triggering DNA damage verification and presumably it is also involved in detecting and binding to structurally damaged nucleotides in ssDNA. Furthermore, XPA interacts with most NER proteins. Next, the single strand gap is filled through the activity of DNA polymerases including DNA Pol δ, ε or κ. Finally, GG-NER is completed by sealing the nick via DNA ligase I or XRCC1-DNA ligase 3. The Transcription coupled repair pathway (TC-NER) has the ability to detect DNA alterations in the transcribed strand during transcription elongation. The stalling or arrest of RNA polymerase II triggers the localization of CSB to the DNA damage site. This protein is highly regulated during this process due to the function of the deubiquitin ligase USP7, which protects CSB from CSA-dependent degradation. Furthermore, CSB plays a crucial function in the CRL4^{CSA} complex engagement and coordinates the events of RNA polymerase stalling and chromatin remodeling via p300 and HMGN1. After the removal of RNA polymerase II from the damaged site the strand can be cleaved and the lesion cleared and repaired as described above in the GG-NER sub-pathway. The below Table 1 lists proteins known to be involved in NER:

TABLE 1
proteins known to be involved in NER

| List of | |
|---|---|
| NER genes | Name |
| XPC | Xeroderma pigmentosum, complementation group C |
| RAD23B | UV excision repair protein RAD23 homolog B |
| CETN2 | Centrin 2 |
| RAD23A | UV excision repair protein RAD23 homolog A |
| XPA | Xeroderma pigmentosum, complementation group A |
| DDB1 | DNA damage-binding protein 1 |
| DDB2 (XPE) | DNA damage-binding protein 2 (Xeroderma |
| | pigmentosum, complementation group E) |
| RPA1 | Replication protein A1 |
| RPA2 | Replication protein A2 |
| RPA3 | Replication protein A3 |
| ERCC3 (XPB) | Excision Repair Cross-Complementing Rodent Repair |
| | Deficiency, Complementation Group 3 (Xeroderma |
| | pigmentosum, complementation group B) |
| ERCC2 (XPD) | Excision Repair Cross-Complementing Rodent Repair |
| | Deficiency, Complementation Group 2 (Xeroderma |
| | pigmentosum, complementation group D) |
| GTF2H1 | General Transcription Factor IIH Subunit 1 |
| GTF2H2 | General Transcription Factor IIH Subunit 2 |
| GTF2H3 | General Transcription Factor IIH Subunit 3 |
| GTF2H4 | General Transcription Factor IIH Subunit 4 |
| GTF2H5 (TTDA) | General Transcription Factor IIH Subunit 5 |
| CDK7 | Cyclin Dependent Kinase 7 |
| CCNH | Cyclin H |
| MNAT1 | Menage A Trois 1 |
| ERCC5 (XPG) | Excision Repair Cross-Complementing Rodent Repair |
| | Deficiency, Complementation Group 5 (Xeroderma |
| | Pigmentosum, Complementation Group G) |
| ERCC1 | Excision Repair Cross-Complementing Rodent Repair |
| | Deficiency, Complementation Group 1 |
| ERCC4 (XPF) | Excision Repair Cross-Complementing Rodent Repair |
| | Deficiency, Complementation Group 4 (Xeroderma |
| | Pigmentosum, Complementation Group F) |
| LIG1 | DNA Ligase 1 |
| ERCC8 (CSA) | Excision Repair Cross-Complementing Rodent Repair |
| | Deficiency, Complementation Group 8 (Cockayne |
| | Syndrome WD Repeat Protein CSA) |
| ERCC6 (CSB) | Excision Repair Cross-Complementing Rodent Repair |
| | Deficiency, Complementation Group 6 (Cockayne |
| | Syndrome Group B Protein) |
| UVSSA | UV Stimulated Scaffold Protein A |
| (KIAA1530) | |
| XAB2 (HCNP) | XPA Binding Protein 2 |
| MMS19 | MMS19 Homolog, Cytosolic Iron-Sulfur Assembly |
| | Component |
| USP7 | Ubiquitin-specific-processing protease 7 |
| XPV | Xeroderma Pigmentosum Variant Type Protein |
| RFC | Replication factor C |
| PCNA | Proliferating cell nuclear antigen |

Based on the use of knock out cell lines, in particular cell lines absent of XPA, XPC, ERCC8 (CSA), ERCC6 (CSB), or XPV(POLH), it was shown that the sulfonylurea compounds of the present invention, in particular Acetohexamide or derivatives, have a general protective effect on NER-deficient cell lines.

Accordingly, the present invention provides sulfonylurea compounds for use in the treatment and/or amelioration of a disease that is associated with UV-induced DNA damage, wherein the subject to be treated expresses enzymatically active mutY homolog (MUTYH), and wherein the sulfonylurea compound has the structure of formula I and wherein the disease that is associated with UV-induced DNA damage is a disease that is associated with NER deficiency characterized by at least one mutation in at least one of the NER pathway genes shown in Table 1. In a preferred embodiment, the disease that is associated with NER deficiency is Xeroderma pigmentosum (XP), Cockayne syndrome (CS), UV-sensitive syndrome (UVSS), Trichothiodystrophy (TTD) or cerebro-oculo-facioskeletal syndrome (COFS).

In this regard, the terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease related to an undesired immune response from occurring in a subject which may be predisposed to the disease; (b) inhibiting the disease, i.e. arresting its development; (c) relieving the disease, i.e. causing regression of the disease; or (d) alleviating symptoms associated with the disease.

Thus, the present invention, in one embodiment, provides sulfonylurea compounds for use in the treatment and/or amelioration of a disease that is associated with UV-induced DNA damage, wherein the subject to be treated expresses enzymatically active mutY homolog (MUTYH), and wherein the sulfonylurea compound has the structure of formula I and wherein the disease that is associated with UV-induced DNA damage is a disease that is associated with NER deficiency, and wherein the sulfonylurea compound alleviates symptoms associated with NER deficiency. In a preferred embodiment of the invention, the symptoms associated with NER deficiency are UV sensitivity, UV-irritation, UV-induced DNA damage, UV-induced cell death, the development of cancer, neurological symptoms, premature ageing, and/or developmental defects. In this respect, the development of cancer particularly relates to the development of melanocyte and keratinocyte malignancy, and/or multiple basal cell carcinomas, invasive squamous cell carcinomas and melanomas. Neurological symptoms and developmental defects particularly include hyporeflexia, progressive mental retardation, sensorineural deafness, spasticity, seizures, myelinopathy, microcephaly, very short stature and/or many other characteristics associated with severe neurodevelopmental abnormalities and premature aging. Premature ageing relates to a phenotype of accelerated ageing that is exhibited by patients at a young age.

The present invention also relates to a pharmaceutical composition for use in the treatment and/or amelioration of a disease that is associated with UV-induced DNA damage, wherein the subject to be treated expresses enzymatically active MUTYH, and wherein the pharmaceutical composition comprises the sulfonylurea compound of the invention for the use according to the invention; and optionally a pharmaceutically acceptable carrier.

A “subject” for the purposes of the present invention is used to include both humans and other animals, particularly mammals, and other organisms. Thus, the methods are applicable to both human therapy and veterinary applications. In the preferred embodiment the patient or subject is a mammal, and in the most preferred embodiment the patient or subject is a human.

The expression “pharmaceutical composition” is meant to refer, for the purposes of the present invention, to a therapeutically effective amount of the active ingredient, i.e. the sufonylurea compound of the invention, optionally, together with a pharmaceutically acceptable carrier or diluent.

It embraces compositions that are suitable for the curative treatment, the control, the amelioration, an improvement of the condition or the prevention of a disease or disorder in a human being or a non-human animal. Thus, it embraces pharmaceutical compositions for the use in the area of human or veterinary medicine.

The compounds of the present invention and as described herein in the various embodiments and the pharmaceutical compositions containing said compounds may be administered topically to body surfaces and thus be formulated in a form suitable for topical administration or may be administered orally.

The pharmaceutical compositions provided herein in the various embodiments may also be administered as controlled-release compositions, i.e. compositions in which the active ingredient is released over a period of time after administration. For example, the sulfonylurea compounds of the invention or the pharmaceutical composition of the invention can be released over a longer period of time of, for example, 5, 6, 7, 8, 9 or 10 hours. Controlled- or sustained-release compositions include formulation in lipophilic depots (e.g. fatty acids, waxes, oils). In another embodiment, the composition is an immediate-release composition, i.e. a composition in which all the active ingredient is released immediately after administration.

Suitable dosages of the pharmaceutical compositions according to the invention and as described herein in the various embodiments will vary depending upon the condition, age and species of the subject, and can be readily determined by those skilled in the art. Such dosage will be adjusted to the individual requirements in each particular case including the specific compound(s) being administered, the route of administration, the condition being treated, as well as the patient being treated. However, the compounds can also be administered as depot preparations (implants, slow-release formulations, etc.) weekly, monthly or at even longer intervals. A particular preparation is a plaster, patch or the like. In such cases the dosage will be much higher than the daily one and has to be adapted to the administration form, the body weight and the concrete indication. The appropriate dosage can be determined by conducting conventional model tests, preferably animal models. The daily dosage can be administered as a single dose or in divided doses.

An effective dose of active ingredient(s) depends at least on the nature of the condition being treated, toxicity, whether the compound(s) is being used prophylactically (lower doses) or against an active condition, the method of delivery, and the pharmaceutical formulation, and will be determined by the clinician using conventional dose escalation studies.

In a particular embodiment, the pharmaceutical composition of the invention as described herein in the various embodiments or aspects is administered daily over an extended period of time to an infant. Regular application/administration, in particular daily application, has a beneficial long-term effect of preventing diseases from developing.

Pharmaceutical acceptable carriers are well-known in the art. That is, the person skilled in the art can easily obtain an acceptable carrier for use with the means and methods of the present invention. The pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, and polyvinyl pyrrolidone. The carrier may also comprise any of the substances described in Remington: The Science and Practice of Pharmacy (Gennaro and Gennaro, Eds, 20th edition, Lippincott Williams & Wilkins, 2000); Theory and Practice of Industrial Pharmacy (Lachman et al, eds., 3^{rd} edition, Lippincott Williams & Wilkins, 1986); Encyclopedia of Pharmaceutical Technology (Swarbrick and Boylan, eds., 2nd edition, Marcel Dekker, 2002). The fillers can be chosen from, but are not limited to, powdered cellulose, sorbitol, mannitol, various types of lactose, phosphates and the like.

The polymers can be chosen from, but not limited to, hydrophilic or hydrophobic polymers such as derivatives of cellulose (for example methylcellulose, hydroxypropyl cellulose, hypromellose, ethylcellulose); polyvinylpirolidone (for example povidone, crospovidone, copovidone); polymethacrylates (for example Eudragit RS, RL); lipophilic components (for example glyceryl monostearate, glyceryl behenate); and various other substances such as for example hydroxypropyl starch, polyethylene oxide, carrageenan and the like. Most commonly, hydrophilic swelling polymers of suitable viscosity such as hypromellose are used, preferably in amounts above 5%, and more preferably above 8%. Glidants can be chosen from, but not limited to, colloidal silicon dioxide, talc, magnesium stearate, calcium stearate, aluminium stearate, palmitic acid, stearic acid, stearol, cetanol, polyethylene glycol and the like. Lubricants can be chosen from, but not limited to, stearic acid, magnesium stearate, calcium stearate, aluminium stearate, sodium stearyl fumarate, talc, hydrogenated castor oil, polyethylene glycols and the like.

In a specific embodiment of the invention, the pharmaceutical composition is for topical administration, i.e. it is a topical composition. Topical compositions useful in the present invention involve formulations suitable for topical application to skin. In one embodiment, the composition comprises the sufonylurea compound of the invention and a pharmaceutically-acceptable topical carrier. In one embodiment, the pharmaceutically-acceptable topical carrier is from about 50% to about 99.99%, by weight, of the composition (e.g., from about 80% to about 95%, by weight, of the composition.

The compositions may be made into a wide variety of product types that include but are not limited to lotions, creams, gels, sticks, sprays, shaving creams, ointments, cleansing liquid washes and solid bars, shampoos, pastes, powders, mousses, shaving creams, wipes, patches, nail lacquers, wound dressing, adhesive bandages, hydrogels, films and make-up such as concealers, foundations, mascaras, and lipsticks. These product types may comprise several types of pharmaceutically-acceptable topical carriers including, but not limited to solutions, emulsions (e.g., microemulsions and nanoemulsions), gels, solids, micelles, and liposomes.

The topical compositions useful in the present invention can be formulated as solutions. Solutions typically include an aqueous solvent (e.g., from about 50% to about 99.99%, such as from about 90% to about 99%, by weight of a pharmaceutically acceptable aqueous solvent). Topical compositions useful in the subject invention may be formulated as a solution comprising an emollient. Such compositions preferably contain from about 2% to about 50% of an emollient (s). As used herein, “emollients” refer to materials used for the prevention or relief of dryness, as well as for the protection of the skin. A wide variety of suitable emollients are known and may be used herein. See the International Cosmetic Ingredient Dictionary and Handbook, eds. Wenninger and McEwen, pp. 1656-61, 1626, and 1654-55 (The Cosmetic, Toiletry, and Fragrance Assoc, Washington, D.C., 7^{th} Edition, 1997) (hereinafter “ICI Handbook”) contains numerous examples of suitable materials.

A lotion can be made from such a solution. Lotions typically comprise from about 1% to about 20% (e.g., from about 5% to about 10%) of an emollient (s) and from about 50% to about 90% (e.g., from about 60% to about 80%) of water.

Another type of product that may be formulated from a solution is a cream. A cream typically comprises from about 5% to about 50% (e.g., from about 10% to about 20%) of an emollient (s) and from about 45% to about 85% (e.g., from about 50% to about 75%) of water.

Yet another type of product that may be formulated from a solution is an ointment. An ointment may comprise a simple base of animal or vegetable oils or semi-solid hydrocarbons. An ointment may comprise from about 2% to about 10% of an emollient (s) plus from about 0.1% to about 2% of a thickening agent (s). A more complete disclosure of thickening agents or viscosity increasing agents useful herein can be found in the ICI Handbook pp. 1693-1697. The topical compositions useful in the present invention can also be formulated as emulsions. If the carrier is an emulsion, from about 1% to about 10% (e.g., from about 2% to about 5%) of the carrier comprises an emulsifier (s). Emulsifiers may be nonionic, anionic or cationic. Suitable emulsifiers are disclosed in the ICI Handbook, pp 0.1673-1686.

Lotions and creams can be formulated as emulsions. Typically, such lotions comprise from 0.5% to about 5% of an emulsifier (s). Such creams would typically comprise from about 1% to about 20% (e.g., from about 5% to about 10%) of an emollient (s); from about 20% to about 80% (e.g., from 30% to about 70%) of water; and from about 1% to about 10% (e.g., from about 2% to about 5%) of an emulsifier (s). Single emulsion skin care preparations, such as lotions and creams, of the oil-in-water type and water-in-oil type are well-known in the cosmetic art and are useful in the subject invention. Multiphase emulsion compositions, such as the water-in-oil-in-water type are also useful in the subject invention. In general, such single or multiphase emulsions contain water, emollients, and emulsifiers as essential ingredients.

The topical compositions of this invention can also be formulated as a gel (e.g., an aqueous gel using a suitable gelling agent (s). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprise between about 0.1% and 5%, by weight, of such gelling agents.

The topical compositions of the present invention can also be formulated into a solid formulation (e.g., a wax-based stick, soap bar composition, powder, or a wipe containing powder).

Liposomal formulations are also useful compositions of the subject invention. Examples of liposomes are unilamellar, multilamellar, and paucilamellar liposomes, which may or may not contain phospholipids. Liposomes typically have size from about 50 nm to about 10 microns, such as about 0.1 to about 1 micron. Such compositions can be prepared by first combining the carboxylic acid with a phospholipid, such as dipalmitoylphosphatidyl choline, cholesterol and water. Epidermal lipids of suitable composition for forming liposomes may be substituted for the phospholipid. Examples of such epidermal lipids include, but are not limited to, glyceryl monoesters and diesters, polyethylene fatty ethers, and sterols. The liposome preparation may then incorporated into one of the above carriers (e.g., suspended in a solution, gel, or an oil-in-water emulsion) in order to produce the liposomal formulation.

Micelle formulations are also useful compositions of the subject invention. Such compositions can be prepared using single chain surfactants and lipids.

Micelles typically have size from about 1 nm to about 100 nm, such as from about 10 nm to about 50 nm. The micelle preparation may then incorporated into one of the above carriers (e.g., a gel or a solution) in order to produce the micelle formulation.

The topical compositions useful in the subject invention may contain, in addition to the aforementioned components, a wide variety of additional oil-soluble materials and/or water-soluble materials conventionally used in compositions for use on skin, hair, and nails at their art-established levels.

An effective amount refers to that amount which provides a therapeutic effect for a given condition and administration regimen. In particular, “therapeutically effective amount” means an amount that is effective to prevent, alleviate or ameliorate symptoms of the disease. Determination of a therapeutically effective amount is within the skill of the person skilled in the art. The therapeutically effective amount or dosage of a compound according to this invention can vary within wide limits and may be determined in a manner known in the relevant art. The dosage can vary within wide limits and will, of course, have to be adjusted to the individual requirements in each particular case.

The sulfonylurea compounds of the invention may be formulated for oral administration, for example, with an inert diluent or with an edible carrier, or it may be enclosed in hard or soft shell gelatin capsules, compressed into tablets, or incorporated directly with the food of the diet. For oral therapeutic administration, the active compound may be incorporated with an excipient and used in the form of ingestible tablets, buccal tablets, coated tablets, troches, capsules, elixirs, dispersions, suspensions, solutions, syrups, wafers, patches, and the like.

Tablets, troches, pills, capsules and the like may also contain one or more of the following: a binder such as gum tragacanth, acacia, corn starch or gelatin; an excipient, such as dicalcium phosphate; a disintegrating agent such as corn starch, potato starch, alginic acid and the like; a lubricant such as magnesium stearate; a sweetening agent such as sucrose, lactose or saccharin; or a flavoring agent such as peppermint, oil of wintergreen or cherry flavoring. When the unit dosage form is a capsule, it may contain, in addition to materials of the above type, a liquid carrier. Various other materials may be present as coating, for instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. It may be desirable for material in a dosage form or pharmaceutical composition to be pharmaceutically pure and substantially non toxic in the amounts employed.

Some compositions or dosage forms may be a liquid, or may comprise a solid phase dispersed in a liquid.

In some embodiments, an oral dosage form may comprise a silicified microcrystalline cellulose such as Prosolv. For example, about 20% (wt/wt) to about 70% (wt/wt), about 10% (wt/wt) to about 20% (wt/wt), about 20% (wt/wt) to about 40% (wt/wt), about 25% (wt/wt) to about 30% (wt/wt), about 40% (wt/wt) to about 50% (wt/wt), or about 45% (wt/wt) to about 50% (wt/wt) silicified microcrystalline cellulose may be present in an oral dosage form or a unit of an oral dosage form.

In some embodiments, an oral dosage form may comprise a crosslinked polyvinylpyrrolidone such as crospovidone. For example, about 1% (wt/wt) to about 10% (wt/wt), about 1% (wt/wt) to about 5% (wt/wt), or about 1% (wt/wt) to about 3% (wt/wt) crosslinked polyvinylpyrrolidone may be present in an oral dosage form or a unit of an oral dosage form.

In some embodiments, an oral dosage form may comprise a fumed silica such as Aerosil. For example, about 0.1% (wt/wt) to about 10% (wt/wt), about 0.1% (wt/wt) to about 1% (wt/wt), or about 0.4% (wt/wt) to about 0.6% (wt/wt) fumed silica may be present in an oral dosage form or a unit of an oral dosage form.

In some embodiments, an oral dosage form may comprise magnesium stearate. For example, about 0.1% (wt/wt) to about 10% (wt/wt), about 0.1% (wt/wt) to about 1% (wt/wt), or about 0.4% (wt/wt) to about 0.6% (wt/wt) magnesium stearate may be present in an oral dosage form or a unit of an oral dosage form.

An oral dosage form comprising a sulfonylurea compound of the invention may be included in a pharmaceutical product comprising more than one unit of the or dosage form.

A pharmaceutical product containing oral dosage forms for daily use can contain 28, 29, 30, or 31 units of the oral dosage form for a monthly supply. An approximately 6 week daily supply can contain 40 to 45 units of the oral dosage form. An approximately 3 month daily supply can contain 85 to 95 units of the oral dosage form. An approximately six-month daily supply can contain 170 to 200 units of the oral dosage form. An approximately one year daily supply can contain 350 to 380 units of the oral dosage form.

A pharmaceutical product containing oral dosage forms for weekly use can contain 4 or 5 units of the oral dosage form for a monthly supply. An approximately 2 month weekly supply can contain 8 or 9 units of the oral dosage form. An approximately 6 week weekly supply can contain about 6 units of the oral dosage form. An approximately 3 month weekly supply can contain 12, 13 or 14 units of the oral dosage form. An approximately six-month weekly supply can contain 22 to 30 units of the oral dosage form. An approximately one year weekly supply can contain 45 to 60 units of the oral dosage form.

A pharmaceutical product may accommodate other dosing regimes. For example, a pharmaceutical product may comprise 5 to 10 units of the oral dosage form, wherein each unit of the oral dosage form contains about 40 mg to about 150 mg of the sulfonylurea compound oft he invention. Some pharmaceutical products may comprise 1 to 10 units of the oral dosage form, wherein the product contains about 200 mg to about 2000 mg of the sulfonylurea compound oft he invention. For such a product, each unit of the oral dosage form may be taken daily for 1 to 10 days or 5 to 10 days during a month, such as at the beginning of a month.

Some oral dosage forms comprising a sulfonylurea compound of the invention may have enteric coatings or film coatings.

The present invention also relates to a screening method for identifying a compound that treats and/or ameliorates a disease that is associated with UV-induced DNA damage in a subject that expresses enzymatically active MUTYH, wherein the method comprises contacting a test compound with MUTYH or a cell expressing MUTYH; measuring the expression and/or activity of MUTYH in the presence and absence of said test compound; and identifying a compound that reduces the expression and/or activity of MUTYH as a compound that treats and/or ameliorates a disease that is associated with UV-induced DNA damage in a subject that expresses enzymatically active MUTYH. In one embodiment, the activity of MUTYH is DNA glycosylase activity. In a preferred embodiment, the DNA glycosylase activity of the enzymatically active MUTYH is tested by incubating MUTYH in the presence of the test compound with a radiolabeled oligo, containing DNA lesions, including guanine, 8-oxo-7,8-dihydroguanine or 2-hydroxy-adenine, CPDs and 6-4PPs, and measure cleavage activity in the complementary non-damaged strand. In one embodiment, the amount measured in the screening methods of the present invention is the amount of the MUTYH polypeptide. In this regard, any technique suitable to determine the amount of a polypeptide in a sample may be employed. Preferred methods include immunoblotting, Mass spectrometry based methods, Enzyme-Linked Immunosorbent Assay (ELISA), Flow cytometry based methods (FACS based methods), Immunohistochemistry based methods and/or Immunofluorescence based methods. However, the person skilled in the art is well-aware that alternative methods exist, which may also be employed.

The cell used in the screening method of the present invention is, in one embodiment, a eukaryotic cell. Preferred cells are fibroblasts derived from the subject, or human cell lines such as human haploid cells, including but not limited to HAP1, HeLa, U2OS, HEK293T or other cell lines where MUTYH is functionally active.

In one embodiment, the screening method of the invention additionally comprises a step of comparing the test compound to a control. Using a control may simplify the assessment of whether a test compound is effective for the desired purpose. For example, in one embodiment, the control is an inactive test compound, wherein said inactive test compound is a compound that does not reduce the expression and/or activity of MUTYH. A negative control can be Dimethyl sulfoxide (DMSO). A positive control can be Acetohexamide.

In one embodiment of the screening method provided herein, the test compound is a small molecule of a screening library; or a peptide of a phage display library, of an antibody fragment library, or derived from a cDNA library.

A test compound identified in the screening methods of the present invention to reduce the expression and/or activity of MUTYH is classified as a compound that treats and/or ameliorates a disease that is associated with UV-induced DNA damage in a subject that expresses enzymatically active MUTYH, whereby the disease that is associated with NER deficiency is Xeroderma pigmentosum (XP), Cockayne syndrome (CS), UV-sensitive syndrome (UVSS), Trichothiodystrophy (TTD) or cerebro-oculo-facioskeletal syndrome (COFS).

The present invention also relates to a method for monitoring the therapeutic success during the treatment of a disease that is associated with UV-induced DNA damage in a subject, wherein the method comprises measuring in a sample obtained from a test subject the amount and/or activity of MUTYH; comparing said amount and/or activity with reference data corresponding to the amount and/or activity of MUTYH of at least one reference subject; and predicting therapeutic success based on the comparison of said amount and/or activity with reference data corresponding to the amount and/or activity of MUTYH of at least one reference subject. In a preferred embodiment of the monitoring method of the invention, the amount of MUTYH measured is the amount of enzymatically active MUTYH polypeptide. In a further preferred embodiment of the monitoring method of the invention, the test subject has expressed enzymatically active MUTYH before the treatment started.

It is furthermore preferred in the monitoring methods of the present invention that in the sample which was obtained from the test subject before the treatment started, the amount of the enzymatically active MUTYH polypeptide is at least 80, 85, 90, 91, 92, 93, 94, 95, 96, 97, 98, 99 or 100% of the amount of the enzymatically active MUTYH polypeptide of a sample obtained from a healthy reference subject.

In one embodiment of the monitoring method of the invention, the test subject is a human being who receives medication for a disease that is associated with NER deficiency.

In the monitoring method of the present invention, the reference data can correspond to the amount and/or activity of MUTYH in a sample of at least one reference subject. In a preferred embodiment of the monitoring method of the present invention, the at least one reference subject has a disease that is associated with NER deficiency but did not receive medication for this disease; and wherein when predicting therapeutic success based on the comparison of said amount and/or activity with reference data corresponding to the amount and/or activity of MUTYH of at least one reference subject a decreased amount and/or activity of MUTYH of the test subject as compared to the reference data indicates therapeutic success in the treatment of a disease that is associated with NER deficiency. A decreased amount and/or activity of MUTYH may, in one embodiment, mean that the amount and/or activity of MUTYH in the sample of the test subject is 0 to 10, 0 to 20, 0 to 30, 0 to 40, 0 to 50, 0 to 60, 0 to 70, 0 to 80 or 0 to 90% of the amount and/or activity of MUTYH in the sample of at least one reference subject. Preferably, at least one reference subject has a disease that is associated with NER deficiency and has received medication for this disease; and wherein when predicting therapeutic success based on the comparison of said amount and/or activity with reference data corresponding to the amount and/or activity of MUTYH of at least one reference subject an identical or similar amount and/or activity of MUTYH of the test subject as compared to the reference data indicates therapeutic success in the treatment of a disease that is associated with NER deficiency.

In an alternative embodiment, at least one reference subject does not have a disease that is associated with NER deficiency; and wherein when predicting therapeutic success based on the comparison of said amount and/or activity with reference data corresponding to the amount and/or activity of MUTYH of at least one reference subject an identical or similar amount and/or activity of MUTYH of the test subject as compared to the reference data an identical or similar amount and/or activity of MUTYH of the test subject as compared to the reference data indicates therapeutic success in the treatment of a disease that is associated with NER deficiency.

The identical or similar amount and/or activity of MUTYH means, preferably, that the amount and/or activity of MUTYH in the sample of the test subject is 10, 20, 30, 40, 50, 60, 70, 80 or 90-100 or 110%, preferably 90-110% of the amount and/or activity of MUTYH in the sample of the at least one reference subject.

The invention furthermore relates to a method for identifying a subject which responds to a treatment with a sulfonylurea compound according to the invention, wherein the method comprises measuring the expression and/or activity of MUTYH in a sample obtained from a test subject; and identifying a subject which comprises enzymatically active MUTYH as a responder to a treatment with a sulfonylurea compound according to the invention.

Preferably, the subject has a disease that is associated with UV-induced DNA damage such as the diseases defined herein. Furthermore, it is preferred that the amount of enzymatically active MUTYH in the sample of the test subject is at least as high has the amount of enzymatically active MUTYH of a sample of a healthy reference subject.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While aspects of the invention are illustrated and described in detail in the drawings and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below. The invention also covers all further features shown in the figures individually, although they may not have been described in the previous or following description. Also, single alternatives of the embodiments described in the figures and the description and single alternatives of features thereof can be disclaimed from the subject matter of the other aspect of the invention.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention. A number of documents including patent applications, manufacturer's manuals and scientific publications are cited herein. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

FIG. 1. Acetohexamide alleviates the UV sensitivity of NER-deficient cells

A. Schematic representation of the experimental setup used to perform the high-throughput drug screen, where drugs were used at five-times maximal plasma concentration (CLOUD; CeMM library of unique drugs). B. Bubble plot displaying the drugs used plotted against cell viability. Light grey bubbles indicate wildtype (WT) cells, dark grey bubbles indicate XPA-deficient cells (ΔXPA). The size of the bubbles indicates significance, displayed as −log₁₀ (p-value). C. Chemical structure of acetohexamide. D. Dose-response curve of WT and ΔXPA cells treated with or without 0.5 mM acetohexamide for 6 hours, followed by UV irradiation. Survival was assessed after 3 days, using CellTiter-Glo. Displayed is the relative viability obtained by normalizing the raw data of the DMSO control to acetohexamide treated cells. Error bars indicate SEM (n=3). E. Colony formation using the same conditions indicated in (D), where cells were kept in culture for 10 days following UV irradiation, following which they were fixed and stained.

FIG. 2. Acetohexamide enhances the clearance of cyclobutane pyrimidine dimers in NER-deficient cells

A. Dose-response curve of WT and ΔXPA cells treated with or without 0.5 mM acetohexamide for 6 hours, followed by illudin S treatment. Survival was assessed after 3 days using CellTiter-Glo. B. Colony formation of WT fibroblasts (BJ) and XPA-patient derived fibroblasts (XPA^{Δ/Δ}) treated with or without 0.5 mM acetohexamide for 6 hours, followed by UV irradiation (as indicated) and then kept in culture for 10 days. C. WT BJ and XPA^{Δ/Δ} fibroblastoid cells were treated with 0.5 mM acetohexamide for 6 hours, irradiated with 15 J/M² and then fixed and immunostained with an anti-cyclobutane pyrimidine dimer (CPD) antibody at the indicated times. Nuclear DNA was counterstained with DAPI. Scale bar, 10 μm. D. Scatter plot displaying the quantification of CPD intensities per nucleus of WT and XPA^{Δ/Δ} cells in the presence or absence of 0.5 mM acetohexamide, of more than 100 cells. Red lines within each column represent median intensities. A.u.=arbitrary unit.

FIG. 3. Loss of MUTYH mimics acetohexamide function A. Bubble plot displaying the percentage of rescue, defined as the difference in survival of a given cell line treated with acetohexamide compared to untreated, following UV irradiation. The light grey, dark grey bubbles highlight ΔMUTYH, ΔXPA and cells respectively, and black bubbles indicate the rest of the knockout cell lines and WT HAP1. The size of the bubbles indicates the significance as log₁₀ (p-value). BER: base excision repair; NER: nucleotide excision repair; DSBR; double-strand break repair; MMR: mis-match repair; FA: Fanconi aneamia; DR; direct reversal; TLS; translesion synthesis. B. Survival of WT and MUTYH-deficient (ΔMUTYH) cells with or without 0.5 mM acetohexamide treatment, followed by UV irradiation, as assessed after 3 days using CellTitre-Glo. Loss of MUTYH protein was confirmed by immunoblotting using an anti-MUTYH antibody. ACTIN was used as a loading control. C. Deletion of MUTYH in WT HAP1 or an XPA-deficient background was confirmed by immunoblotting using an anti-MUTYH antibody. TUBULIN was used as a loading control. D. Clonogenic survival of WT, ΔXPA or ΔXPA-MUTYH cells irradiated with UV at the indicated doses or left untreated. Cells were fixed and stained 10 days later.

FIG. 4. Loss of MUTYH, or use of acetohexamide, corrects UV sensitivity and defective clearance of cyclobutane pyrimidine dimers in NER-deficient cells, without accumulation of chromosomal instability

A. WT HAP1 cells were treated with or without 0.5 mM acetohexamide for 6 hours, then released into compound-free media for the indicated time points and immunoblotted with an anti-MUTYH antibody. ACTIN was used as a loading control. B. WT HAP1 cells were either treated with 0.5 mM acetohexamide alone or with 10 μM of the proteasome inhibitor MG132 for 6 hours and analyzed by immunoblotting using an anti-MUTYH antibody. C. Left panel: colony formation of the WT, ΔXPA or ΔXPA-MUTYH HAP1 cells treated with or without 0.5 mM acetohexamide for 6 hours, followed by 15 J/M² UV irradiation and then kept in culture for 10 days. Right panel: Macroscopic colonies were stained with crystal violet and quantified. D. WT, ΔXPA or ΔXPA-MUTYH HAP1 cells were treated with 15 J/M² UV or left untreated, then kept in culture for the indicated recovery times and analyzed by dot blot for the presence of CPDs within genomic DNA. E. Number of chromosomal abnormalities per metaphase spread of ΔXPA or ΔXPA-MUTYH HAP1 exposed to different doses of UV irradiation. Data in (E) represented as mean±SEM. F. Survival of WT and ΔMULE HAP1 cells exposed to UV irradiation at different doses and assessed after 3 days, using CellTitre-Glo.

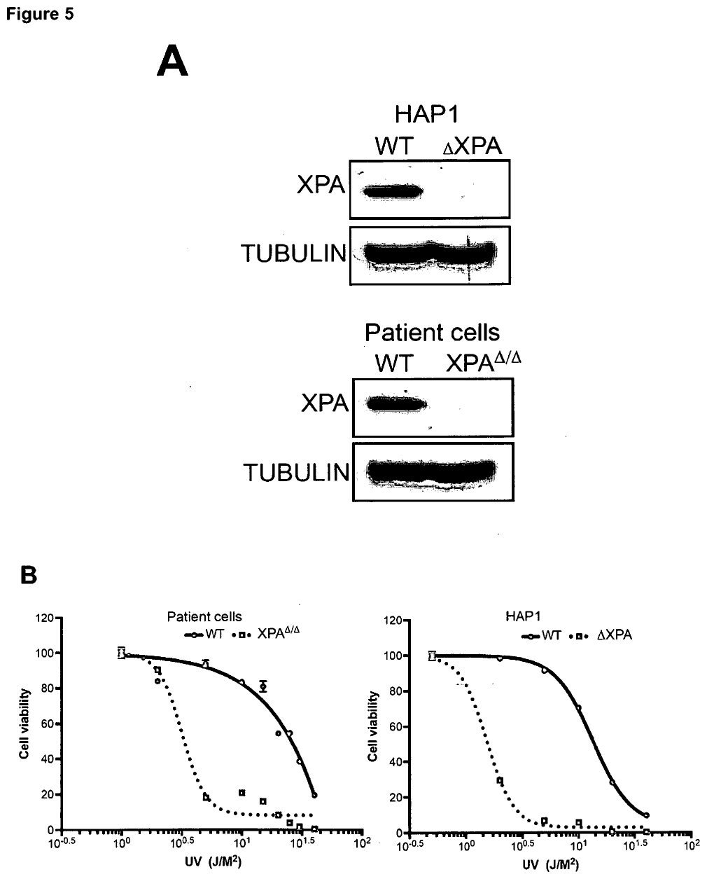

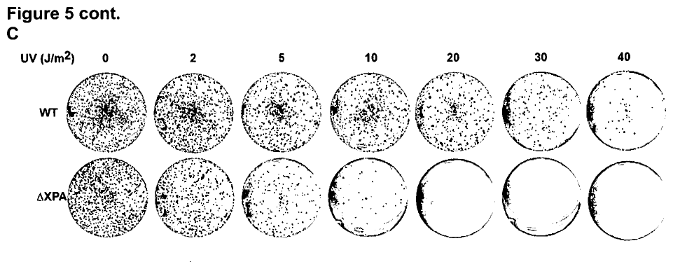

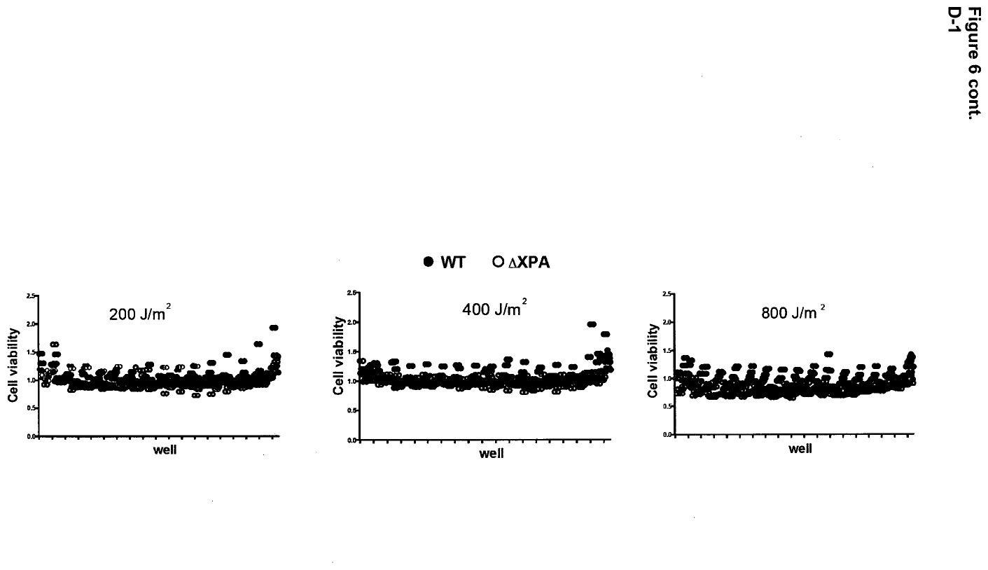

FIG. 5. Generation of XPA-deficient HAP1 cells and experimental optimization of the high-throughput drug screen

A. Immunoblots of whole cell extracts of HAP1 WT and ΔXPA cells as well as fibroblastoid WT BJ cells and XPA patient-derived fibroblasts (XPA^{Δ/Δ}). TUBULIN was used as a loading control. B. Survival of HAP1 WT and ΔXPA cells as well as WT and patient-derived fibroblasts (XPA^{Δ/Δ}) was assessed 3 days following UV exposure using CellTitre-Glo. C. Colony formation of WT and ΔXPA cells irradiated with UV at different doses as indicated and kept in culture for 10 days. D. WT and ΔXPA cells were seeded in 384-well plates and irradiated at different UV doses, as indicated.

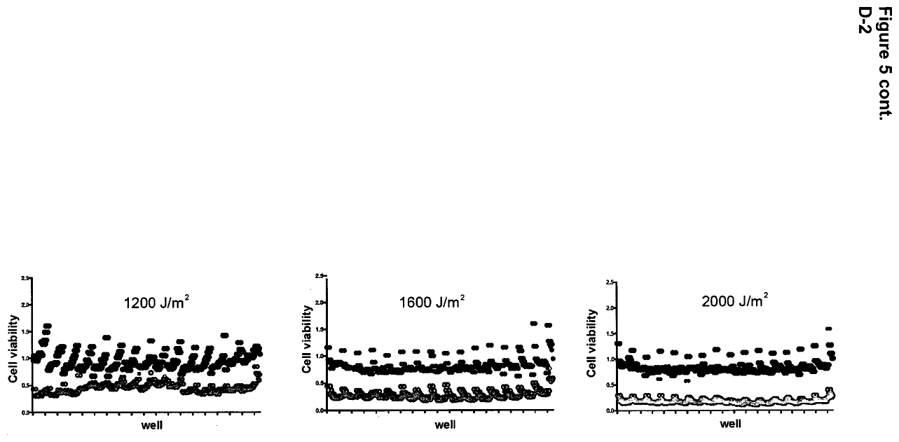

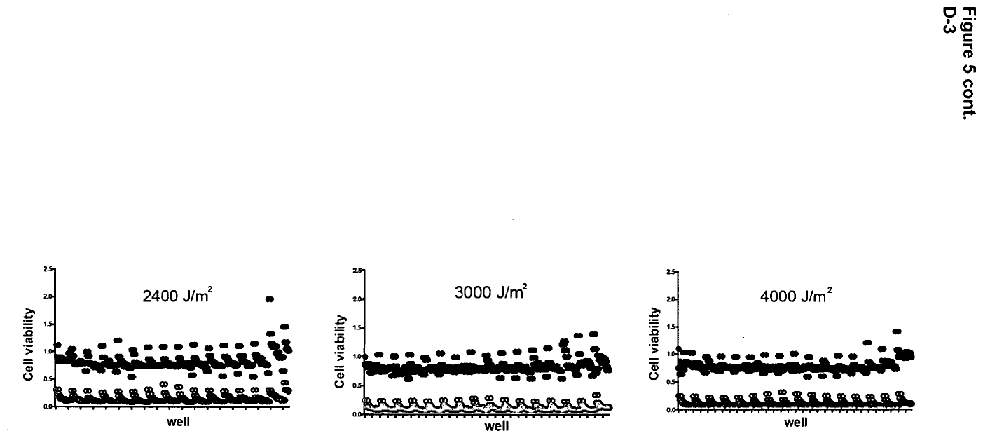

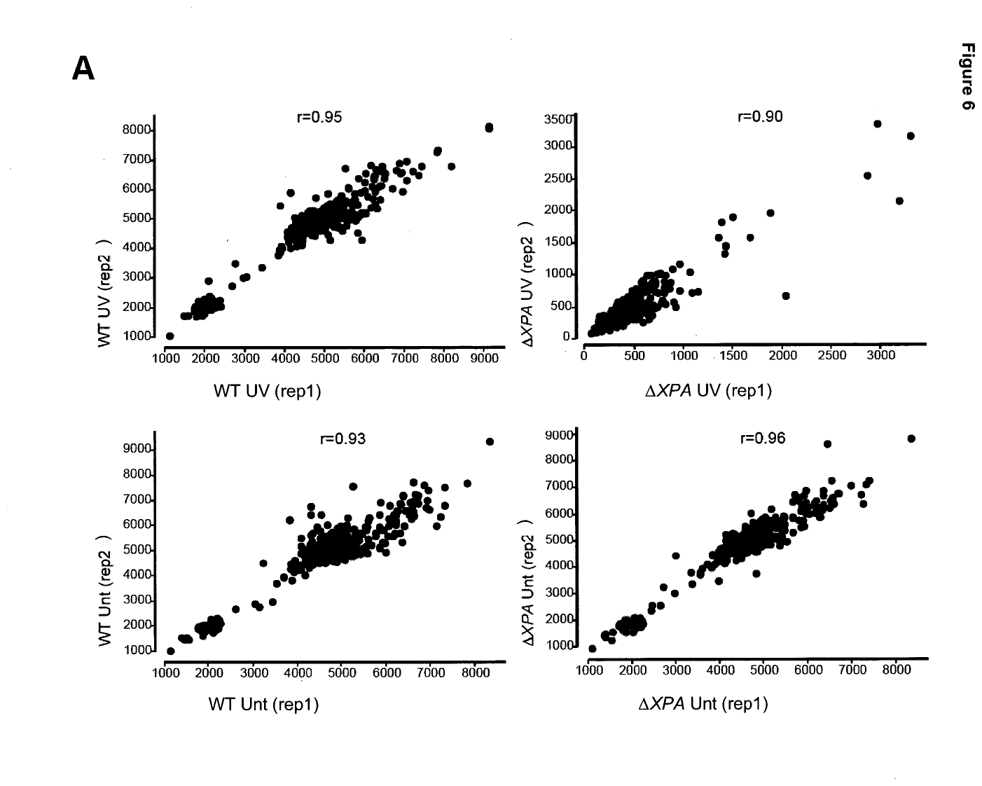

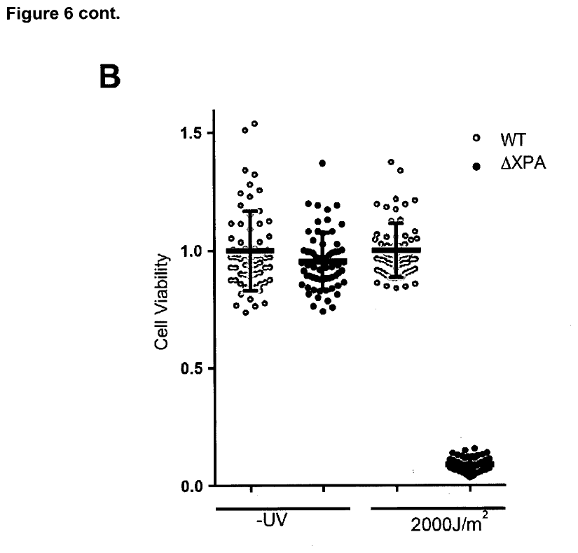

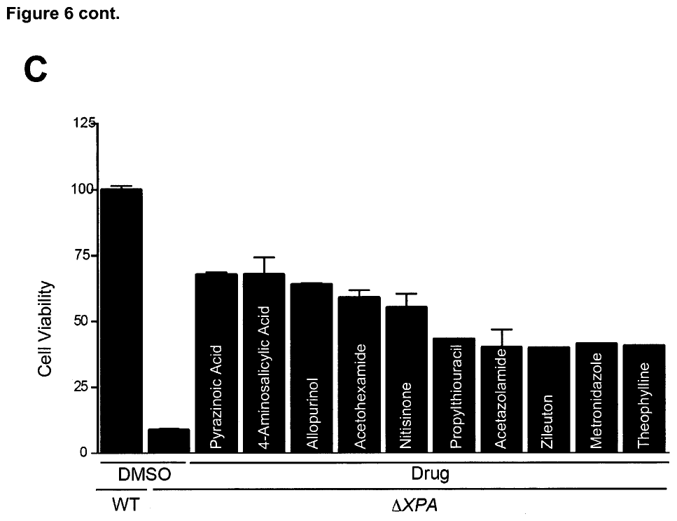

FIG. 6. High-throughput drug screen for agents that alleviate UV sensitivity of NER-deficient cells

A. Spearman's rank correlation coefficient to determine the experimental reproducibility of the two biological replicates for the high-throughput drug screen performed on WT and ΔXPA cells after UV irradiation or under untreated conditions. B. Separation between DMSO control-treated samples after 2,000 J/M² UV irradiated or untreated. C. Top 10 drugs that showed an alleviation of cell death of ΔXPA cells of more than 40%, compared to wildtype (WT) cells.

FIG. 7. Acetohexamide alleviates UV and illudin S sensitivity of ΔXPA cells due to enhanced clearance of CPDs

A. Dose-response curve of WT and ΔXPA cells treated with or without 0.5 mM acetohexamide for the indicated times, followed by UV irradiation. Survival was assessed after 3 days, using CellTiter-Glo. Displayed is the relative viability obtained by normalizing the raw data of the DMSO control to acetohexamide treated cells. Error bars indicate SEM (n=3). B. Clonogenic survival of WT and ΔXPA cells treated with 0.5 mM acetohexamide for 6 hours or left untreated, then exposed to illudin S for 10 days, as indicated. C. WT and ΔXPA cells treated with or without 0.5 mM acetohexamide for the indicated times, followed by 15 J/M² irradiation and analyzed by dot blot for the presence of CPDs in genomic DNA. DNA was counterstained with methylene blue (MB) as a loading control. D. Quantification of the intensities of C.

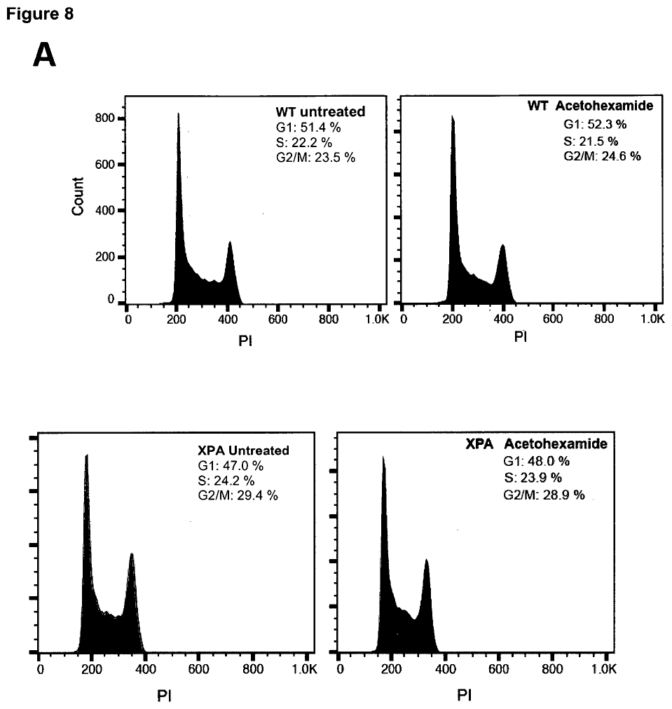

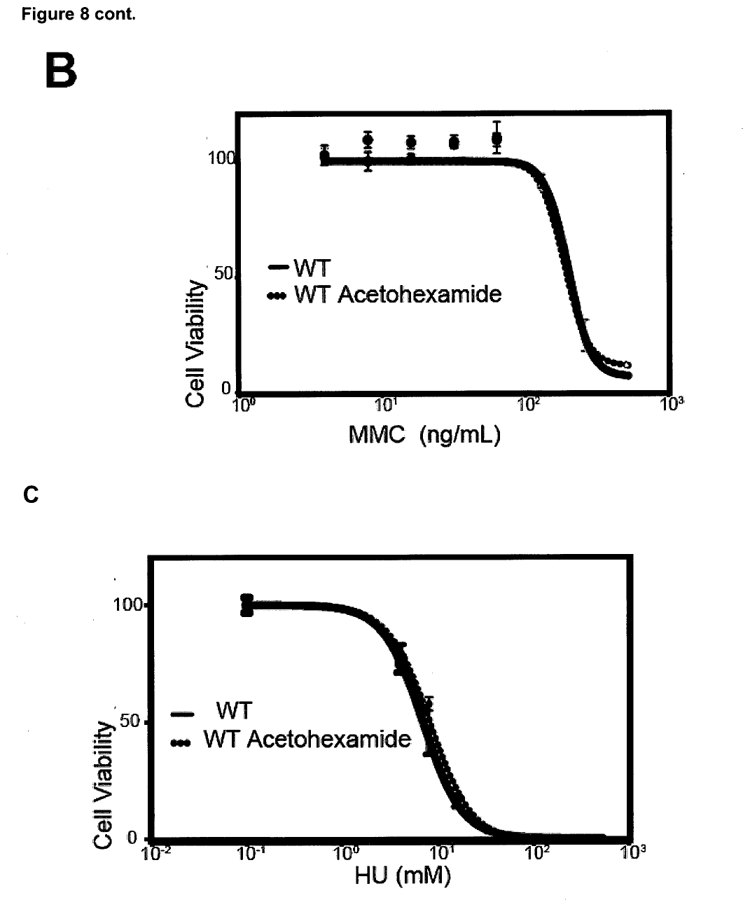

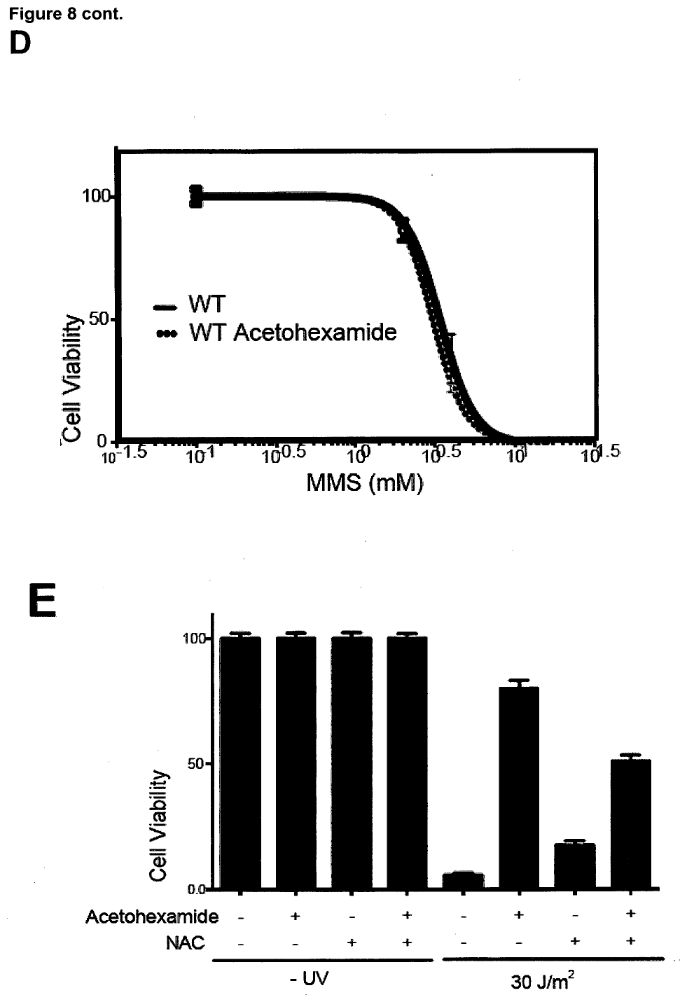

FIG. 8. Acetohexamide does not function by altering cell cycle, apoptosis or by quenching reactive oxygen species

A. WT and ΔXPA cells were either treated with DMSO or 0.5 mM acetohexamide for 6 hours. Cell cycle profiles were determined using propidium iodide (P1) staining followed by FACS analysis. B-D. Survival of WT HAP1 cells treated with either DMSO or 0.5 mM acetohexamide for 6 hours, followed by exposure to the indicated DNA damaging agents (MMC: mitomycin C; HU: hydroxyl urea; MMS: methyl methanesulfonate). Survival was assessed after 3 days using CellTitre-Glo. E. Cell viability of WT cells treated either with 0.5 mM acetohexamide or 30 μM N-acetylcysteine (NAC) for 6 hours, followed by 30 J/M² UV exposure.

FIG. 9. Acetohexamide does not function via SUR1 inhibition, to correct viability after UV

A. Fragments Per Kilobase Million (FPKM) for SUR1 and GAPDH in HAP1 WT cells compared, from RNA sequencing. B. mRNA expression of SUR1 transcript assessed by quantitative reverse transcription PCR in WT and ΔXPA cells with or without 0.5 mM acetohexamide treatment for 6 hours, followed by 15 J/M² UV irradiation and subsequent recovery as indicated. Expression of GAPDH was used as a reference. Error bars indicate SEM (n=3).

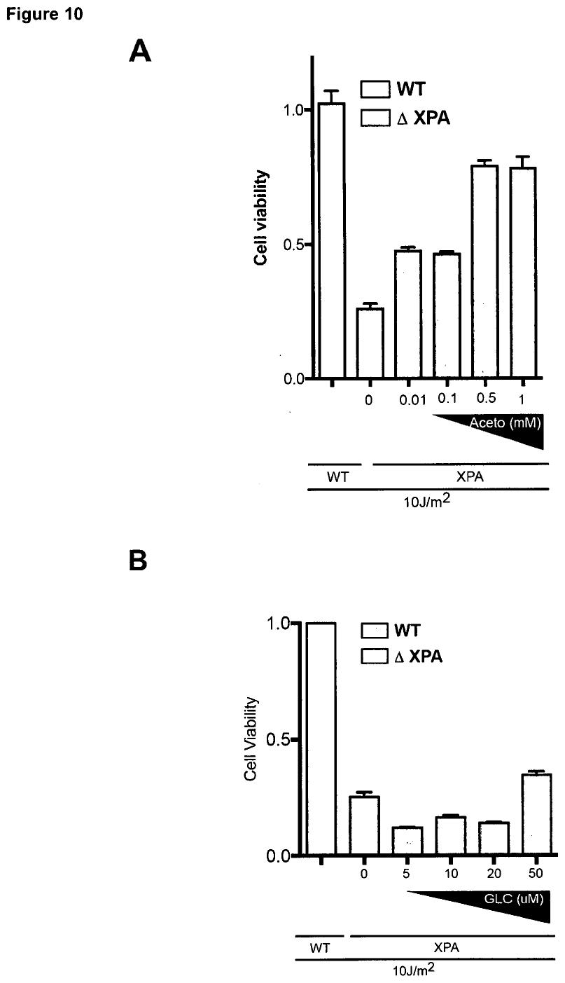

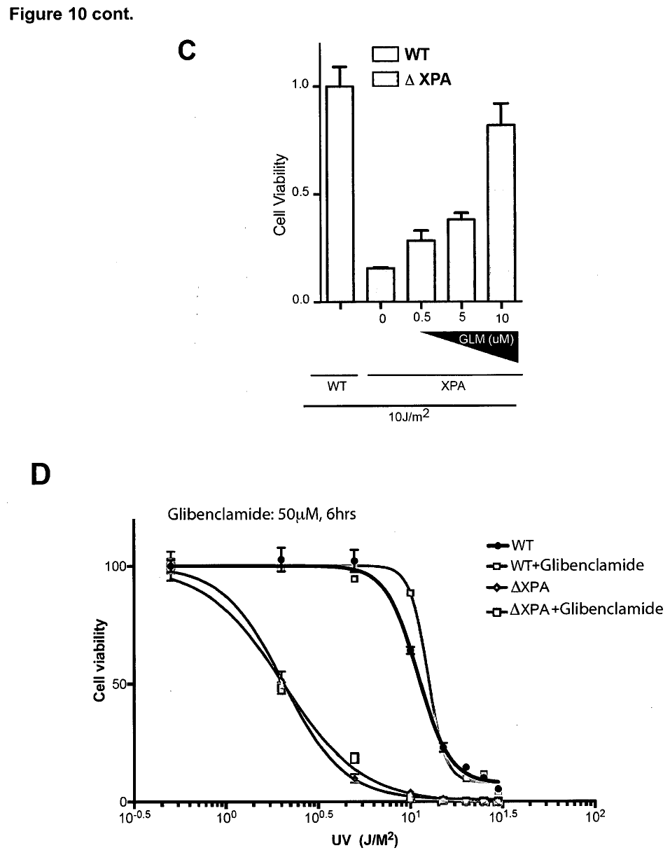

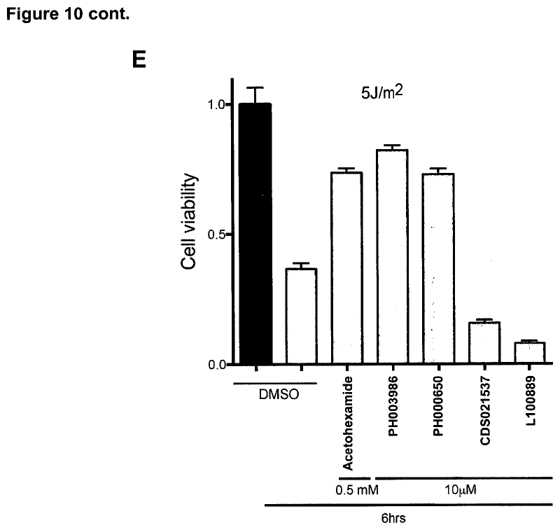

FIG. 10. Investigation of sulfonylurea compounds that correct the UV sensitivity of NER-deficient cells

A-C. Cell viability of ΔXPA cells treated with different concentrations of acetohexamide (aceto), gliclazide (GLC) and glimepiride (GLM) for 6 hours, followed by 10 J/M² of UV irradiation. D. Survival of WT and ΔXPA cells treated with or without 50 μM glibenclamide for 6 hours, followed by UV exposure. Survival was assessed after 3 days using CellTitre-Glo. E. Cell viability of ΔXPA cells treated with 10 μM of different derivatives of acetohexamide for 6 hours, followed by UV irradiation with 5 J/M².

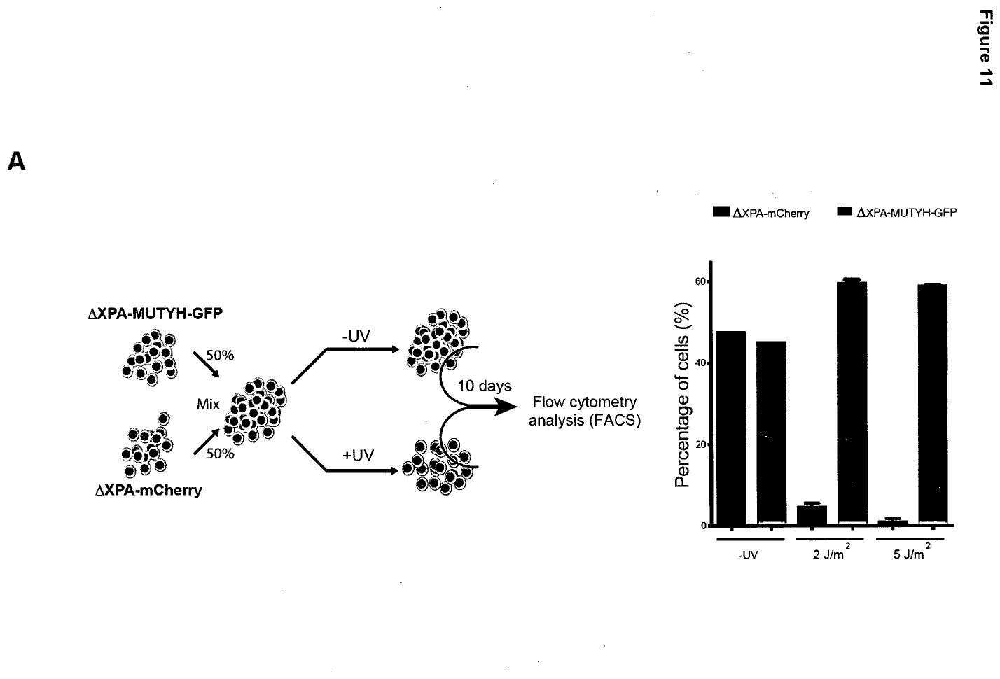

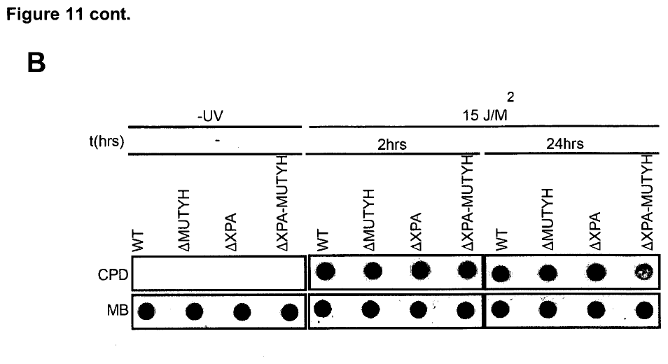

FIG. 11. Loss of MUTYH enhances clearance of cyclobutane pyrimidine dimers and corrects UV sensitivity of XPA-deficient cells

A. ΔXPA (mCherry⁺) and ΔXPA-MUTYH (GFP⁺) were mixed equally and then UV irradiated with different doses, followed by FACS analysis after 10 days. B. WT, ΔMUTYH, ΔXPA ΔXPA-MUTYH HAP1 cells treated with 15 J/M², followed by recovery for the indicated times. Genomic DNA was and analyzed for the presence of CPDs by dot blot. DNA was counterstained with methylene blue (MB) as a loading control.

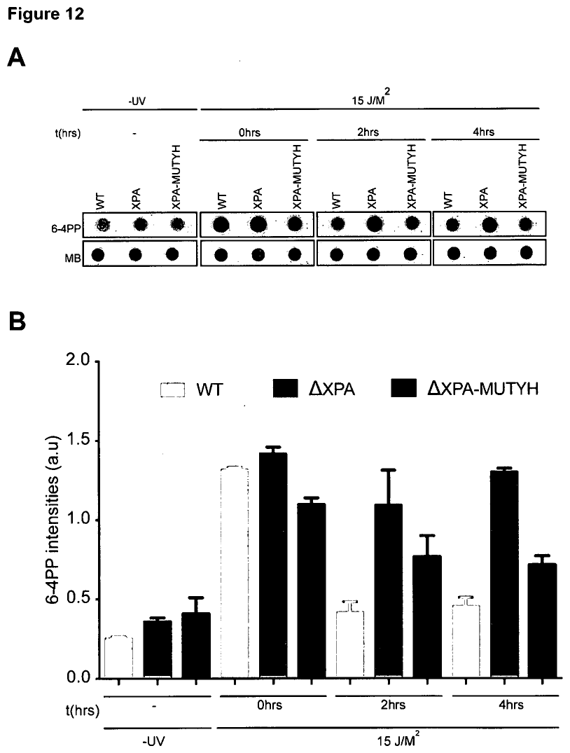

FIG. 12. Clearance of 6-4 PPs

A. WT, ΔXPA or ΔXPA-MUTYH HAP1 cells were treated with 15 J/M2 UV or left untreated, then kept in culture for the indicated recovery times following which genomic DNA was extracted and 6-4 pyrimidine-pyrimidone photoproducts (6-4PPs) were analyzed by dot blot.

Total DNA was counterstained with methylene blue (MB) as a loading control.

B. Quantification of A.

The data shows that MUTYH loss in XPA deficient cells allows clearance of 6-4PPs hence alleviating DNA repair defects of NER deficient cells with the regard of repairing 6-4PP.

### EXAMPLE 1

### Screening of Compounds

An in-house drug library of around 300 compounds representing all structurally distinct Food and Drug Administration (FDA) approved compounds was used to allow for potential drug repurposing. First, an NER-deficient cell line was generated by making a frameshift mutation in XPA, one of the central components of NER that functions in both TC-NER and GG-NER, utilizing CRISPR-Cas9 in the human near haploid cell line HAP1 (denoted ΔXPA) (FIG. 5A). As expected, and similar to an XPA patient-derived fibroblastoid cell line (denoted)XPA^{Δ/Δ}, ΔXPA cells displayed enhanced sensitivity to UV irradiation (FIG. 5B-C). Next, ΔXPA and wildtype cells were exposed to the drug library (with each drug used at five times maximal plasma concentration) for 24 hours, followed by UV exposure (at a dose selected to kill ΔXPA cells but not wildtype cells) (FIGS. 1A and 5D). The compounds were scored based on their efficiency to improve cellular survival of ΔXPA cells, compared to wildtype cells (FIG. 1B). A correlation greater than 0.9 was obtained between the biological replicates with sufficient separation between ΔXPA and wildtype cells (FIG. 6A-B).

Ten compounds were identified that showed more than a 40% correction of survival of ΔXPA cells compared to wildtype cells (FIG. 6C). Eight of the ten compounds were excluded for further analysis due to their ability to block UV-induced DNA damage and hence indirectly increase cellular survival. One of the two remaining compounds was acetohexamide (FIG. 1C), an anti-diabetic drug that belongs to the first generation of sulfonylurea drugs [Joseph et al (2010), J Chromatogr B Analyt Technol Biomed Life Sci, 878: 2775-81].

### EXAMPLE 2

### Functional Analysis of Acetohexamide, and Further Sulfonylurea Compounds

Acetohexamide alleviated the UV sensitivity of ΔXPA cells almost to the level of wildtype cells both in a short-term dose response assay (FIG. 1D) and in a long-term colony formation assay (FIG. 1E). To determine whether acetohexamide could also correct cellular survival following other sources of DNA crosslinking damage, we exposed wildtype and ΔXPA cells to illudin S, a genotoxin that induces bulky adducts that are repaired by NER (FIG. 2A and FIG. 7B). We observed that indeed, acetohexamide could increase cellular survival following illudin S treatment. We also confirmed that acetohexamide could alleviate UV-induced cell death of XPA^{Δ/Δ} patient-derived cells (FIG. 2B), indicating that the mode of rescue is not cell type specific. Next we determined whether incubation with acetohexamide leads to a clearance of UV-induced lesions by measuring the levels of CPDs, the most predominant lesions induced by UV and representing approximately 75% of UV lesions. As expected wildtype cells that are NER proficient were able to clear CPDs 24 hours post UV irradiation, whereas, NER-deficient XPA^{Δ/Δ} cells continued to show elevated levels of CPDs at 24 hours post UV irradiation. However, strikingly acetohexamide led to the clearance of CPDs in XPA^{Δ/Δ} cells, suggesting that acetohexamide enhances the ability of NER-deficient cells to clear CPDs lesions. Importantly, acetohexamide did not affect the initial amount of CPDs (FIGS. 2C-D). The same observation was also made for HAP1 cell lines (FIGS. 7C-D).

Next, three additional sulfonylureas that stimulate insulin release via ATP-dependent K⁺ channels were tested including gliclazide (GLC), glimepiride (GLM) and glibenclamide. Only glimepiride showed a protective effect of LXPA cells against UV (FIG. 10A-D). Two additional derivatives of sulfonylurea showed a potent effect within the range of μM amounts (FIG. 10E), proving that the invention is not limited to the specific structure of Acetohexamide.

### EXAMPLE 3

### Mode of Action of Acetohexamide

To gain insight into the mode of action of acetohexamide, cell cycle profiles upon exposure to the compound were assessed. There was no difference between wildtype or ΔXPA cells upon acetohexamide treatment, ruling out an effect on cell cycle phase (FIG. 8A). To exclude the possibility that acetohexamide has a general anti-apoptotic effect, wildtype cells were treated with a variety of different DNA damaging agents including the DNA crosslinking agent mitomycin C (MMC), hydroxyurea (HU), which depletes cellular pools of ribonucleosides thereby inducing replication stress, and the alkylating agent methyl methanesulfonate (MMS). Acetohexamide did not increase cellular survival following exposure to MMC, HU and MMS. Thus acetohexamide does not act as an anti-apoptotic agent following DNA damage (FIG. 8B-D). Furthermore, the potent antioxidant N-acetylcysteine (NAC) showed a very minor effect in alleviating UV-induced sensitivity compared to acetohexamide (FIG. 8E), suggesting that acetohexamide is not exerting its effect simply by quenching reactive oxygen species.

Sulfonylureas, including acetohexamide, target ATP sensitive potassium channels and play a prominent role in regulating insulin secretion. Sulfonylureas are reported to block the inward rectifier of Kir6.2 subunits through their binding to SUR1 (for sulfonylurea receptor 1), leading to membrane depolarization, Ca²⁺ influx, and subsequent insulin release [Proks et al (2002), Diabetes, 51 Suppl 3: S368-76] [Burke et al (2008), Circ Res, 102: 164-76]. However, expression profiling via RNA sequencing analysis did not detect any SUR1 transcript in ΔXPA cells (FIG. 9A). Moreover, SUR1 was not expressed in ΔXPA cells following UV irradiation or acetohexamide treatment (FIG. 9B). Based on these data, SUR1 as the target of acetohexamide was disregarded within this context.

Since acetohexamide enhanced the clearance of CPDs in NER-deficient cells, it was speculated that its mode-of-action could be via one the known DNA excision repair pathways. Thus, a panel of 20 DNA repair-deficient cell lines was prepared using CRISPR-Cas9, representing all DNA repair pathways. Pol kappa (POLK) was selected to represent translesion synthesis (TLS) polymerases since it has roles in the repair synthesis step of NER. Subsequently, these cell lines (as well as two wildtype controls) were treated with acetohexamide and exposed to UV irradiation (FIG. 3A). The ‘percentage of rescue’ was defined as the difference in survival of a given cell line treated with acetohexamide compared to untreated, following UV irradiation (FIG. 3A). Acetohexamide had a comparable protective effect against UV-induced damage on all the knockout cell lines tested (and also to wildtype cells) but had no effect on cells lacking MUTYH. This suggested that acetohexamide and MUTYH have a related function. It also provided further evidence that acetohexamide has a general effect on protecting cells against UV-induced DNA damage.

### EXAMPLE 4

### The Role of MUTYH

MUTYH is a DNA glycosylase that catalyzes the excision of the adenine mis-paired with 8-oxo-guanine in the base excision repair (BER) pathway. Thus, MUTYH is an unusual glycosylase since it removes an undamaged base situated opposite a DNA lesion, instead of removing the damaged base [Markkanen et al (2013), Front Genet, 4: 18]. It was surprisingly found that loss of MUTYH conferred resistance to UV irradiation compared to wildtype cells, similar to the effect of acetohexamide treatment. Furthermore, pre-incubation with acetohexamide did not have a noticeable effect on survival (FIG. 3B), further suggesting that acetohexamide and loss of MUTYH have functionally related effects. To test for a role of MUTYH in NER more directly, it was analyzed whether MUTYH deletion could alleviate the sensitivity of ΔXPA cells by generating a double knockout (ΔXPA-MUTYH) using CRISPR-Cas9 (FIG. 3C). Enhanced cellular survival of ΔXPA-MUTYH cells following UV exposure compared to ΔXPA cells was observed (FIG. 3D). To confirm this finding ΔXPA cells were labeled with mCherry and ΔXPA-MUTYH with GFP. Next these cell lines were mixed in equal amounts and then irradiated with UV at different doses. After 10 days in culture the cells were analyzed by flow cytometry. While the ΔXPA-mCherry cells were no longer detected, the ΔXPA-MUTYH-GFP cells were, indicating that loss of MUTYH confers cellular resistance to UV (FIG. 11A).

It was thus shown that both acetohexamide and loss of MUTYH protect both wildtype and NER-deficient cells from UV-induced cell death. To determine whether acetohexamide works via MUTYH its effect on MUTYH protein levels was first analyzed. It was found that acetohexamide treatment of wildtype cells led to a decrease in MUTYH protein levels in a proteasome dependent manner (FIG. 4A-B). This suggested that acetohexamide is indeed exhibiting its functions by promoting the degradation of MUTYH. In further support of this, acetohexamide treatment of the double knockout ΔXPA-MUTYH did not lead to a further increase in survival upon UV treatment (FIG. 4C). Importantly, ΔXPA-MUTYH cells cleared CPDs more efficiently compared to ΔXPA cells 24 hours post UV irradiation (FIGS. 4D and 11B), suggesting that the observed toxicity in ΔXPA cells following UV activity is MUTYH dependent.

Next it was determined whether the alleviation of UV sensitivity in ΔXPA-MUTYH cells has an effect on chromosomal instability. Hence chromosomal abnormalities in ΔXPA cells were compared to ΔXPA-MUTYH cells, following UV exposure. ΔXPA-MUTYH cells displayed a significant reduction in chromosomal abnormalities after UV irradiation compared to ΔXPA cells (FIG. 4E). Taken together, MUTYH loss has protective effects on genomic stability in ΔXPA cells following UV irradiation.

Collectively, acetohexamide, an anti-diabetic drug, can alleviate the sensitivity of NER-deficient cells and enhance the repair of UV lesions through degradation of MUTYH. It has been shown that MUTYH is ubiquitinated by the E3 ligase MULE, thereby reducing its protein levels and subsequent recruitment to chromatin [Dorn et al (2014), J Biol Chem, 289: 7049-58]. Hence loss of MULE sensitizes cells to UV irradiation due to an accumulation of MUTYH protein. Indeed, MULE-deficient cells (ΔMULE) also showed enhanced sensitivity to UV irradiation (FIG. 4F). Taken together, the data shows that acetohexamide functions by inhibiting a deubiquitin ligase that in turn leads to increased MUTYH ubiquitination by MULE and subsequent degradation.

### EXAMPLE 5

Use of Sulfonylurea Compounds

Sulfonylurea compounds such as acetohexamide, via MUTYH degredation, unmask a NER-independent mechanism for removing UV-induced DNA damage. This pathway leads to the clearance of CPDs and hence improves cellular survival of NER-deficient cells following UV exposure. This occurs in the absence of increased chromosomal instability. MUTYH is a DNA glycosylase that excises adenine bases mispaired with guanine, 8-oxo-7,8-dihydroguanine or 2-hydroxy-adenine and has not previously been implicated in the removal of UV-induced lesions.

Acetohexamide, which is already clinically approved to treat diabetes mellitus type 2, one of its derivatives, or a MUTYH inhibitor, can be used to alleviate symptoms associated with a deficiency in NER. This opens up new therapeutic approaches for the treatment of the many NER-associated diseases. This approach is beneficial for the range of syndromes characterised by UV sensitivity, including XP, CS, UVSS and TTD. The sulfonylurea compounds provided herein that function specifically in the brain or the skin, or that are applied topically, provide therapeutic opportunities for alleviating NER deficiency diseases such as XP CS, UVSS and TTD.

### EXAMPLE 6

### Materials and Methods

### Cell Culture and Reagents

HAP1 cells were cultured in Iscove's Modified Dulbecco's Medium (Gibco). The XPA patient-derived fibroblast cell line was purchased from Coriell Biorepository (GM04429) and cultured in MEM (Gibco), as were BJ cells. All cells were grown in the presence of 10% fetal bovine serum (FBS) (Thermo Fisher Scientific) and 1% penicillin-streptomycin (Sigma-Aldrich) at 37° C. with 5% CO₂ and 3% O₂. Illudin S, hydroxyurea (HU), mitomycin C (MMC), methyl methanesulfonate (MMS), acetohexamide, N-acetylcysteine, gliclazide, glimepiride, glibenclamide, L100889, PH003986, CDS021537 and PH000650 were purchased from Sigma-Aldrich.

### Generation of CRISPR-Cas9 Edited Cell Lines

The DNA repair knockout cell lines were generated in collaboration with Horizon Genomics. Briefly, HAP1 cells were transfected with plasmids expressing Cas9 (pX165 from the Zhang lab), a guide RNA and a blasticidin resistance gene using Xfect (clontech). Cells were then treated with 20 μg/ml blasticidin for 24 hours to eliminate untransfected cells. After allowing the cells to recover for 5 to 7 days from antibiotic selection, clonal cell lines were isolated by limiting dilution. Subsequently, the genomic DNA was isolated using Direct PCR-Cell Kit (PeqLab) and the region targeted by the gRNA was PCR amplified and analyzed by Sanger sequencing. Finally, clones with frameshift mutations were selected for further analysis.

### High-Throughput Drug Screen

Fifty nL of compound per well was transferred into 384-well plates (Corning 3712) from DMSO stock plates using acoustic transfer (Labcyte Echo 520). Wildtype and XPA-deficient HAP1 cells (at an amount of 1,000 cells) were seeded in 50 μl media into the compound-containing plates. After 24 hours, cells were UV irradiated with 2,000 J/M². Three days later cell viability was determined using Cell Titer-Glo (Promega). The screen was performed in duplicate. For data analysis, the percentage of control was calculated and the signal of the DMSO irradiated sample was used to set values to 0% while the DMSO non-irradiated sample was used to set the values to 100%. Hits were defined based whether they corrected survival by more than 40% and the signal was 3 standard deviation away from the DMSO treated conditions.

### Karyogram Analysis

Preparation of metaphases was carried out by standard methods. Briefly, dividing cells were blocked in metaphase by adding 0.1 μg/ml Colcemid (Gibco, Thermo Fisher) for 30-60 minutes. Afterwards cells were treated for 20 minutes with hypotonic solution and fixed using Methanol/Acetic Acid (one part Acetic Acid and three parts Methanol). Then cells were dropped onto slides, dried at 42° C. for about 20 minutes and then incubated at 60° C. overnight. Chromosomes were digested in 2.5% trypsin/NaCl solution for 30 seconds and incubated for about 5 seconds in ice-cold 0.9% NaCl solution. Finally, slides were stained in buffered Giemsa stain solution for 3 minutes. Karyotyping was done using the “MetaSystems Ikaros” software version 5.3.18.

Dose Responses and UV Treatment Dose-response curves for the DNA damage agents including mitomycin C (MMC), methyl methanesulfonate (MMS), hydroxyurea (HU), neocarzinostatin (NCS) and illudin S were performed in 96-well plates by seeding 1,000 cells/well in triplicates. The next day, compounds at different concentrations were added and 3 days later, cell viability was assessed using Cell Titer-Glo (Promega)

For UV irradiation, cells were washed with PBS, trypsinized, counted and distributed in equal number then irradiated with different doses of UV as indicated. Finally, 1,000 cells were re-distributed into 96 well plates. After 72 hours, survival was measured using Cell Titer-Glo (Promega). Cells were irradiated with UVC using the UVP CX-2000 device (254 nm, Fisher Scientific).

### Colony Formation Assay

Cells were treated with UV at different doses with or without drug pre-treatment and then seeded into 6-well plates, at a density of 1,000 cells/well, in duplicates for 2 weeks until visible colonies were formed. Then, the medium was removed, colonies were washed with PBS and fixed using 3.7% paraformaldehyde (PFA) for 1 hour. Subsequently, the PFA was removed and colonies were stained with 0.1% crystal violet in 5% ethanol solution for 1 hour. Next, the staining solution was removed and the wells were washed, imaged and quantified using CellProfiler.

### Cell Cycle Analysis

Cells were treated with either DMSO or acetohexamide as indicated. Cell cycle stages were identified using propidium iodide (PI) staining. Briefly, cells were harvested, resuspended in PBS and fixed overnight with cold 70% ethanol. After centrifugation, ethanol was removed and cells were resuspended in PBS containing 1 μg/mL RNase A and 1 μg/mL P. Finally, cells were analyzed on a FACScalibur flow cytometer. Following cell acquisition, analysis was performed using FlowJo software (Tree Star).

### Quantitative Reverse Transcription PCR

WT and ΔXPA HAP1 cells were harvested and RNA was isolated using phenol-chlorophorm extraction. After treatment with 1 μl DNase (Sigma), the cDNA was transcribed using SuperScript III Reverse Transcriptase (Invitrogen). An amount of 1 μg of cDNA template was used for the qRT-PCR using SYBR Green qPCR Mastermix (Qiagen). Analysis was performed in triplicates using GAPDH as a control gene. The PCR was performed on a 7900HT Fast Real-Time PCR System (Applied Biosystems). The following primers were used:

SUR1:
5′-AGCTGAGAGCGAGGAGGATG-3′;
5′-CACTTGGCCAGCCAGTAGTC-3′,
GAPDH:
5′-AGAACATCATCCCTGCATCC-3′;
5′-ACATTGGGGGTAGGAACAC-3′.

### Protein Extracts and Immunoblotting

Cells were lysed in lysis buffer composed of RIPA lysis buffer supplemented with protease inhibitors (Sigma) and phosphatase inhibitors (Sigma, NEB). After sonication and centrifugation of the lysates, they were heated with reducing sample buffer. Protein samples were separated by SDS-PAGE (3-8% or 4-12% gradient gels; Invitrogen) and then transferred onto nitro-cellulose membranes. All primary antibodies were used at 1:1,000 dilution and secondary antibodies at 1:5,000. Antibodies used were: XPA (14607S; Cell Signaling), MUTYH (ab55551; Abeam), TUBULIN (3873, Cell Signaling) (07-164; Millipore) and R-ACTIN (A 5060A, Sigma).

### Immunofluorescence for Cyclobutane Pyrimidine Dimers and Associated Microscopy

For measurement of cyclobutane pyrimidine dimers (CPDs), cells were seeded onto coverslips (VWR) in 5 cm dishes. On the following day, cells were treated as indicated. Next, they were washed twice with PBS and fixed with 4% paraformaldehyde (PFA) for 10 minutes at room temperature (RT), then permeabilized with 0.5% Triton X-100 in PBS for 5 minntes at RT. After 3 steps of washing with PBS, DNA was denatured with 2M HCL for 30 minutes at room temperature, followed by blocking with 10% FBS in PBS for 30 minutes at 37° C. The primary anti-CPDs and secondary antibodies (anti-CPDs: TDM-2, Cosmo Bio; secondary antibody: Alexa Fluor 488 goat anti-mouse, Invitrogen) were diluted in PBS (1:1,000) and incubated on cells for 30 minutes at 37° C., with five washes (PBS) performed between individual steps. Finally, cells were stained with DAPI (Sigma-Aldrich) for 20 minutes at RT in the dark. Cell images were taken on a deconvolution microscope (Leics). Quantification was performed using CellProfiler.

### Dot Blot for Cyclobutane Pyrimidine Dimers

The amount of cyclobutane pyrimidine dimers (CPDs) in DNA was quantified using an immuno-dot blot assay with the CPD-specific monoclonal antibody TDM-2 (Cosmo Bio). Genomic DNA was extracted using QIAamp DNA mini kit (Qiagen), then the genomic DNA was denatured in TE buffer (10 mM Tris-CL and 1 mM EDTA, pH 7.5) by boiling for 5 minutes and subsequently 50 ng of genomic DNA was dot-blotted in triplicate onto a nitrocellulose membrane. The DNA was then fixed by baking the membrane for 2 hours at 80° C. The membranes were blocked for 1 hour in TBS, 0.2% Tween 20 (TBS-T) containing 5% (w/v) milk. After washing in TBS-T for 15 minutes, the membranes were incubated overnight at room temperature at 4° C. with the monoclonal antibody TDM-2 (anti CPD monoclonal antibody, Cosmo Bio) using a dilution of 1:1,500 in TBS-T. After washing 5 times for 15 minutes, membranes were incubated for 1 hour with anti-mouse secondary antibody diluted 1:2,500 in phosphate-buffered saline (Invitrogen). Signals were detected using Amersham ECL (GE Healthcare Life Sciences DNA was counterstained with methylene blue as a loading control.

### Statistical Analysis

Data are expressed as ±standard error of the mean (SEM) unless otherwise stated.

1. A sulfonylurea compound for use in the treatment and/or amelioration of a disease that is associated with UV-induced DNA damage, wherein the subject to be treated expresses enzymatically active mutY homolog (MUTYH), and wherein the sulfonylurea compound has the structure of Formula I: 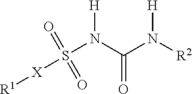
wherein
X is phenylene which is optionally substituted with —NH₂,
R¹ is selected from —C₁₋₆ alkyl, —C(O)—C₁₋₆ alkyl, —(C₁₋₆ alkylene)-C(O)—NH—R³ and —(C₁₋₆ alkylene)-NH—C(O)—R³,
wherein R³ are independently selected from monocyclic unsaturated heterocyclyl containing from 1 to 3 nitrogen atoms and optionally one or two additional heteroatoms selected from S and O, wherein the heterocyclyl optionally has one or two substituents selected from oxo (═O), -halogen, —C₁₋₆ alkyl and −O—C₁₋₆ alkyl; and
R² is selected from C₅₋₇ cycloalkyl which is optionally substituted with one or two independently selected from C₁₋₆ alkyl.

2. A pharmaceutical composition for use in the treatment and/or amelioration of a disease that is associated with UV-induced DNA damage, wherein the subject to be treated expresses enzymatically active MUTYH, and wherein the pharmaceutical composition comprises
(i) the sulfonylurea compound for the use according to claim 1; and
(ii) optionally a pharmaceutically acceptable carrier.

3. The sulfonylurea compound for the use according to claim 1, or the pharmaceutical composition for the use according to claim 2, wherein the sulfonylurea compound is
(i) acetohexamide or a derivative thereof; or
(ii) glimepiride or a derivative thereof.

4. The sulfonylurea compound for the use according to claim 1 or 3, or the pharmaceutical composition for the use according to claim 2 or 3, wherein enzymatically active MUTYH is wild type MUTYH or MUTYH with increased activity.

5. The sulfonylurea compound for the use according to any one of claims 1, 3 and 4, or the pharmaceutical composition for the use according to any one of claims 2-4, wherein the enzymatically active MUTYH is a polypeptide comprising or consisting of
(i) the amino acid sequence of any one of SEQ ID NOs: 1 to 6;
(ii) an amino acid sequence having at least 80% identity to an amino acid sequence of (i), wherein the polypeptide has DNA glycosylase activity;
(iii) the amino acid sequence of an enzymatically active fragment of SEQ ID NO: 1; or
(iv) an amino acid sequence having at least 80% identity to an amino acid sequence of (iii), wherein the polypeptide has DNA glycosylase activity.

6. The sulfonylurea compound for the use according to any one of claims 1 and 3-5, or the pharmaceutical composition for the use according to any one of claims 2-5, wherein the activity of the enzymatically active MUTYH is at least 80% of the activity of a polypeptide consisting of the amino acid sequence of SEQ ID NO: 1.

7. The sulfonylurea compound for the use according to any one of claims 1 and 3-6, or the pharmaceutical composition for the use according to any one of claims 2-6, wherein in a sample obtained from the subject the expression amount of MUTYH is at least 80% of the expression amount of MUTYH in a sample obtained from a healthy reference subject.

8. The sulfonylurea compound for the use according to any one of claims 1 and 3-7, or the pharmaceutical composition for the use according to any one of claims 2-7, wherein the sample is a sample of the skin.

9. The sulfonylurea compound for the use according to any one of claims 1 and 3-8, or the pharmaceutical composition for the use according to any one of claims 2-8, wherein the sulfonylurea compound decreases the amount of enzymatically active MUTYH.

10. The sulfonylurea compound for the use according to any one of claims 1 and 3-9, or the pharmaceutical composition for the use according to any one of claims 2-9, wherein the sulfonylurea compound inhibits the enzymatic activity of MUTYH, and/or leads to the degradation and/or depletion of MUTYH.

11. The sulfonylurea compound for the use according to any one of claims 1 and 3-10, or the pharmaceutical composition for the use according to any one of claims 2-10, wherein the sulfonylurea compound targets MUTYH directly or indirectly via factors that mediate the inhibition of the enzymatic activity of MUTYH.

12. The sulfonylurea compound for the use according to any one of claims 1 and 3-11, or the pharmaceutical composition for the use according to any one of claims 2-11, wherein the sulfonylurea compound decreases the protein level of enzymatically active MUTYH in a proteasome dependent manner.

13. The sulfonylurea compound for the use according to any one of claims 1 and 3-12, or the pharmaceutical composition for the use according to any one of claims 2-12, wherein the sulfonylurea compound enhances the repair of UV-induced DNA damage.

14. The sulfonylurea compound for the use according claim 13, or the pharmaceutical composition for the use according claim 13, wherein the UV-induced DNA damage are cyclobutane-pyrimidine dimers (CPDs), 6-4 pyrimidine-pyrimidone photoproducts (6-4PPs), Dewar valence isomers and/or Spore photoproducts and other types of UV lesions.

15. The sulfonylurea compound for the use according to any one of claims 1 and 3-14, or the pharmaceutical composition for the use according to any one of claims 2-14, wherein the UV-induced DNA damage is caused by UVA, UVB and/or UVC irradiation.

16. The sulfonylurea compound for the use according to any one of claims 1 and 3-15, or the pharmaceutical composition for the use according to any one of claims 2-15, wherein the sulfonylurea compound alleviates nucleotide excision repair (NER) deficiency and/or enhances NER.

17. The sulfonylurea compound for the use according to claim 16, or the pharmaceutical composition for the use according to claim 16, wherein the NER is transcription-coupled repair (TC-NER) and/or global genome repair (GG-NER).

18. The sulfonylurea compound for the use according to any one of claims 1 and 3-17, or the pharmaceutical composition for the use according to any one of claims 2-17, wherein the disease that is associated with UV-induced DNA damage is a disease that is associated with NER deficiency.

19. The sulfonylurea compound for the use according to claim 18, or the pharmaceutical composition for the use according to claim 18, wherein the disease that is associated with NER deficiency is Xeroderma pigmentosum (XP), Cockayne syndrome (CS), UV-sensitive syndrome (UVSS), Trichothiodystrophy (TTD) or cerebro-oculo-facioskeletal syndrome (COFS).

20. The sulfonylurea compound for the use according to any one of claims 1 and 3-19, or the pharmaceutical composition for the use according to any one of claims 2-19, wherein the sulfonylurea compound alleviates symptoms associated with NER deficiency.

21. The sulfonylurea compound for the use according to claim 20, or the pharmaceutical composition for the use according to claim 20, wherein the symptoms associated with NER deficiency are UV sensitivity, UV-irritation, UV-induced DNA damage, UV-induced cell death, the development of cancer, neurological symptoms, premature ageing, and/or developmental defects.

22. A screening method for identifying a compound that treats and/or ameliorates a disease that is associated with UV-induced DNA damage in a subject that expresses enzymatically active MUTYH, wherein the method comprises:
(a) contacting a test compound with
(a1) MUTYH; or
(a2) a cell expressing MUTYH;
(b) measuring the expression and/or activity of MUTYH in the presence and absence of said test compound; and
(c) identifying a compound that reduces the expression and/or activity of MUTYH as a compound that treats and/or ameliorates a disease that is associated with UV-induced DNA damage in a subject that expresses enzymatically active MUTYH,
and optionally identifying said compound as a compound that treats and/or ameliorates a disease associated with NER deficiency, wherein said disease is preferably selected from Xeroderma pigmentosum (XP), Cockayne syndrome (CS), UV-sensitive syndrome (UVSS), Trichothiodystrophy (TTD) and cerebro-oculo-facioskeletal syndrome (COFS).

23. The screening method of claim 22, wherein the activity that is measured in step (b) is DNA glycosylase activity.

24. The screening method of claim 22 or 23, wherein the amount that is measured in step (b) is the amount of the MUTYH polypeptide.

25. The screening method of any one of claims 22-24, wherein said cell is a eukaryotic cell.

26. The screening method of any one of claims 22-25, which additionally comprises the step of
(b2) comparing the test compound to a control.

27. The screening method of claim 26, wherein in said control an inactive test compound is used, wherein said inactive test compound is a compound that does not reduce the expression and/or activity of MUTYH.

28. The screening method of any one of claims 22-27, wherein said test compound is
(i) a small molecule of a screening library; or
(ii) a peptide of a phage display library, of an antibody fragment library, or derived from a cDNA library.

29. A method for monitoring the therapeutic success during the treatment of a disease that is associated with UV-induced DNA damage in a subject, wherein the method comprises:
(a) measuring in a sample obtained from a test subject the amount and/or activity of MUTYH;
(b) comparing said amount and/or activity with reference data corresponding to the amount and/or activity of MUTYH of at least one reference subject; and
(c) predicting therapeutic success based on the comparison step (b).

30. The monitoring method of claim 29, wherein the amount of the enzymatically active MUTYH polypeptide is measured.

31. The monitoring method of claim 29 or 30, wherein the test subject has expressed enzymatically active MUTYH before the treatment started.

32. The monitoring method of claim 30 or 31, wherein in a sample which was obtained from the test subject before the treatment started, the amount of the enzymatically active MUTYH polypeptide is at least 80% of the amount of the enzymatically active MUTYH polypeptide of a sample obtained from a healthy reference subject.

33. The monitoring method of any one of claims 29-32, wherein the test subject is a human being who receives medication for a disease that is associated with NER deficiency.

34. The monitoring method of any one of claims 29-33, wherein the reference data corresponds to the amount and/or activity of MUTYH in a sample of at least one reference subject.

35. The monitoring method of any one of claims 29-34, wherein the at least one reference subject has a disease that is associated with NER deficiency but did not receive medication for this disease; and wherein in step (c) a decreased amount and/or activity of MUTYH of the test subject as compared to the reference data indicates therapeutic success in the treatment of a disease that is associated with NER deficiency.

36. The monitoring method of claim 35, wherein said decreased amount and/or activity of MUTYH means that the amount and/or activity of MUTYH in the sample of the test subject is 0 to 90% of the amount and/or activity of MUTYH in the sample of the at least one reference subject.

37. The monitoring method of any one of claims 29-34, wherein the at least one reference subject has a disease that is associated with NER deficiency and has received medication for this disease; and wherein in step (c) an identical or similar amount and/or activity of MUTYH of the test subject as compared to the reference data indicates therapeutic success in the treatment of a disease that is associated with NER deficiency.

38. The monitoring method of any one of claims 29-34, wherein the at least one reference subject does not have a disease that is associated with NER deficiency; and wherein in step (c) an identical or similar amount and/or activity of MUTYH of the test subject as compared to the reference data indicates therapeutic success in the treatment of a disease that is associated with NER deficiency.

39. The monitoring method of claim 37 or 38, wherein said identical or similar amount and/or activity of MUTYH means that the amount and/or activity of MUTYH in the sample of the test subject is 90-110% of the amount and/or activity of MUTYH in the sample of the at least one reference subject.

40. A method for identifying a subject which responds to a treatment with a sulfonylurea compound as defined in any one of claims 1-21, wherein the method comprises:
(a) measuring the expression and/or activity of MUTYH in a sample obtained from a test subject; and
(c) identifying a subject which comprises enzymatically active MUTYH as a responder to a treatment with a sulfonylurea compound as defined in any one of claims 1-21.

41. The method of claim 40, wherein the subject has a disease that is associated with UV-induced DNA damage.

42. The method of claim 40 or 41, wherein the amount of enzymatically active MUTYH in the sample of the test subject is at least as high has the amount of enzymatically active MUTYH of a sample of a healthy reference subject.

43. A method for ameliorating/treating a disease a disease that is associated with UV-induced DNA damage, wherein the subject to be treated expresses enzymatically active mutY homolog (MUTYH), wherein the method comprises administering to the subject a sulfonylurea compound having the structure of Formula I: 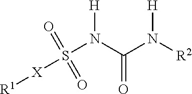
wherein
X is phenylene which is optionally substituted with —NH₂,
R¹ is selected from —C₁₋₆ alkyl, —C(O)—C₁₋₆ alkyl, —(C₁₋₆ alkylene)-C(O)—NH—R³ and —(C₁₋₆ alkylene)-NH—C(O)—R³,
wherein R³ are independently selected from monocyclic unsaturated heterocyclyl containing from 1 to 3 nitrogen atoms and optionally one or two additional heteroatoms selected from S and O, wherein the heterocyclyl optionally has one or two substituents selected from oxo (═O), -halogen, —C₁₋₆ alkyl and —O—C₁₋₆ alkyl; and
R² is selected from C₅₋₇ cycloalkyl which is optionally substituted with one or two independently selected from C₁₋₆ alkyl.
